(19) United States
(12) Patent Application Publication
(10) Pub. No.: US 2023/0253091 A1
(43) Pub. Date: Aug. 10, 2023

(54) DIAGNOSTIC COMMUNICATION SYSTEMS, DIAGNOSTIC DATA COLLECTION KITS, AND METHODS FOR GENERATING AND CONVEYING ORAL CONDITION DATA SETS AND TREATMENT PLANS

(71) Applicant: Iamthinking LLC, Shreveport, LA (US)

(72) Inventors: Christopher C. Cosse, Shreveport, LA (US); Stephen Gillum, Shreveport, LA (US)

(21) Appl. No.: 18/304,278

(22) Filed: Apr. 20, 2023

Related U.S. Application Data
(62) Division of application No. 16/862,992, filed on Apr. 30, 2020.
(60) Provisional application No. 62/841,362, filed on May 1, 2019.

(51) Int. Cl. G16H 20/40; G16H 30/40; G16H 50/20; G16H 70/60; G16H 10/40; G06T 7/00; G06F 16/583

(52) U.S. Cl. CPC G16H 20/40; G06F 16/5854; G06T 7/0014; G16H 10/40; G16H 30/40; G16H 50/20; G16H 70/60; G06T 2207/30036

(57) ABSTRACT

Diagnostic communication systems, diagnostic data collection kits, and methods for generating and conveying oral condition data sets and treatment plans. A method of developing a treatment plan to treat an oral condition of a patient includes receiving an oral condition data set, analyzing the oral condition data set, and generating the treatment plan based on the analysis of the oral condition data set. The oral condition data set is at least partially generated by a patient representative utilizing a data collection kit. A data collection kit for generating an oral condition data set includes at least one diagnostic instrument, instructions describing a method of generating at least a portion of the oral condition data set, and/or packaging that contains the at least one diagnostic instrument. The instructions include instructions for delivering the oral condition data set to a service provider and/or a centralized service.

### RELATED APPLICATIONS

This application claims priority under 35 U.S.C. § 120 to U.S. Pat. Application No. 16/862,992, which was filed on Apr. 30, 2020, which is entitled “DIAGNOSTIC COMMUNICATION SYSTEMS, DIAGNOSTIC DATA COLLECTION KITS, AND METHODS FOR GENERATING AND CONVEYING ORAL CONDITION DATA SETS AND TREATMENT PLANS,” and which claims priority under 35 U.S.C. § 119(e) to U.S. Provisional Pat. Application No. 62/841,362, which was filed on May 1, 2019, and which is entitled “SYSTEMS, DIAGNOSTIC KITS, AND METHODS FOR DELIVERING A DIAGNOSTIC DATA SET REGARDING A PATIENT TO A DOCTOR.” The complete disclosures of the above-identified patent applications are hereby incorporated by reference.

### FIELD OF THE DISCLOSURE

The present disclosure relates to diagnostic communication systems, data collection kits, and methods for generating and conveying oral condition data sets and treatment plans.

### BACKGROUND OF THE DISCLOSURE

Oral and dental conditions such as malocclusion, underbite, overbite, and ankyloglossia often develop at a young age. Such conditions may be treated more effectively if diagnosed at an early age. Other oral and dental conditions may develop in individuals (of any age), sometimes progressively over time, and such individuals may benefit by having the conditions, and treatment options therefor, diagnosed before the conditions become acute and/or otherwise create dental or other health concerns that could have been avoided. Screening for such conditions generally requires that a potential patient meet with a service provider in person, such as at the service provider’s office. However, the inconvenience and/or expense of such in-person screening may restrict potential clients from receiving such a screening. Thus, there exists a need for systems, diagnostic kits, and methods for delivering an oral condition data set regarding a patient to a service provider.

### SUMMARY OF THE DISCLOSURE

Diagnostic communication systems, diagnostic data collection kits, and methods for generating and conveying oral condition data sets and treatment plans are disclosed herein. A method of developing a treatment plan to be performed by a service provider to treat an oral condition of a patient includes receiving an oral condition data set that characterizes the oral condition, analyzing the oral condition data set, and generating the treatment plan. The oral condition data set is generated, at least in part, by a patient representative utilizing a data collection kit. The generating the treatment plan is based, at least in part, on the analyzing the oral condition data set. The method of developing the treatment plan is performed, at least in part, by a service provider representative that includes one or both of the service provider and a service provider proxy.

A data collection kit for generating an oral condition data set that characterizes an oral condition of a patient includes at least one diagnostic instrument configured to facilitate characterizing at least a portion of the oral condition, instructions describing a method of generating at least a portion of the oral condition data set, and/or packaging that contains at least the at least one diagnostic instrument. The instructions include instructions for delivering the oral condition data set to a service provider or a centralized service.

### BRIEF DESCRIPTION OF THE DRAWINGS

FIG. 1 is a schematic diagram representing examples of systems for delivering an oral condition data set and/or a treatment plan according to the present disclosure.

FIG. 2 is a front view of an example of a patient’s mouth according to the present disclosure.

FIG. 3 is a top front perspective view of an example of a data collection kit according to the present disclosure.

FIG. 4 is a top view of an example of a one-piece cheek retractor according to the present disclosure.

FIG. 5 is a side view of an example of a cheek retractor portion according to the present disclosure.

FIG. 6 is a front view of an example of a one-piece cheek retractor during operative use to retract a patient’s cheeks, according to the present disclosure.

FIG. 7 is a top view of an example of an integrated gauge tool according to the present disclosure.

FIG. 8 is a bottom view of the integrated gauge tool of FIG. 7.

FIG. 9 is an illustration of an example of a tracking code according to the present disclosure.

FIG. 10 is a flowchart schematically representing examples of methods for conveying an oral condition data set regarding an oral condition of a patient to a service provider or a centralized service according to the present disclosure.

FIG. 11 is an example of a user interface for conveying instructions to a patient representative according to the present disclosure.

FIG. 12 is an example of a user interface for conveying instructions to a patient representative according to the present disclosure.

FIG. 13 is an example of a user interface for conveying instructions to a patient representative according to the present disclosure.

FIG. 14 is an example of a user interface for conveying an explanatory resource to a patient representative according to the present disclosure.

FIG. 15 is a flowchart schematically representing examples of methods for developing a treatment plan to be performed by a service provider to treat an oral condition of a patient according to the present disclosure.

FIG. 16 is an example of a patient photograph with visual reference points according to the present disclosure.

FIG. 17 is an example of a patient photograph with visual reference points according to the present disclosure.

FIG. 18 is an example of a patient photograph with visual reference points and a diagnostic indicator according to the present disclosure.

### DETAILED DESCRIPTION

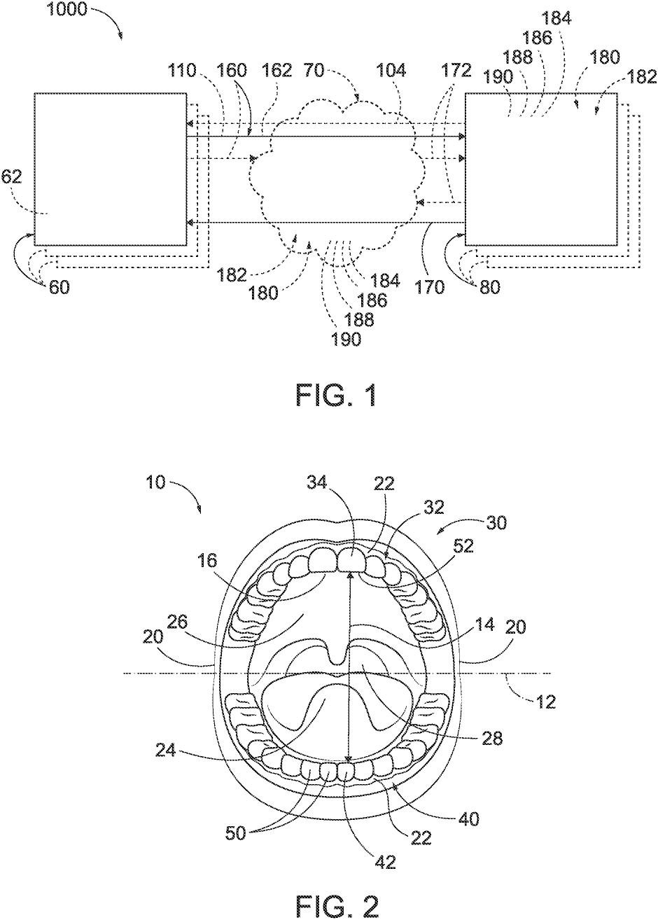

FIGS. 1-18 provide examples of diagnostic communication systems 1000, of diagnostic data collection kits 100, of methods 200 of conveying an oral condition data set regarding a patient, and/or of methods 300 of developing a treatment plan to be performed by a service provider to treat an oral condition of a patient, according to the present disclosure. Elements that serve a similar, or at least substantially similar, purpose are labeled with like numbers in each of FIGS. 1-18, and these elements may not be discussed in detail herein with reference to each of FIGS. 1-18. Similarly, all elements may not be labeled in each of FIGS. 1-18, but reference numerals associated therewith may be utilized herein for consistency. Elements, components, and/or features that are discussed herein with reference to one or more of FIGS. 1-18 may be included in and/or utilized with any of FIGS. 1-18 without departing from the scope of the present disclosure.

In general, in the Figures, elements that are likely to be included in a given (i.e., a particular) embodiment are illustrated in solid lines, while elements that are optional to a given embodiment are illustrated in dashed lines. However, elements that are shown in solid lines are not essential to all embodiments, and an element shown in solid lines may be omitted from a given embodiment without departing from the scope of the present disclosure.

FIG. 1 is a schematic illustration of a diagnostic communication system 1000, such as may be utilized for coordinating information transfer between a patient (or patient representative) and a service provider (or service provider representative), which as discussed herein, may be located remotely from each other. As an example, and as discussed in more detail herein, the service provider may be a provider of medical services, and the patient may be an individual that is seeking, or prospectively seeking, the services of a service provider, or of the particular service provider. As more specific examples, the patient may be an active (e.g., current) patient of the service provider, or the patient may be a potential (e.g., prospective) patient who has not yet committed to engaging the services of the service provider.

When evaluating the necessity and/or benefit of pursuing and/or enacting a medical service, it generally is preferable that the service provider and the patient have the ability to interact directly (e.g., in person) to facilitate the service provider observing the patient’s medical condition and subsequently forming a diagnosis and/or treatment plan. However, in some cases, such in-person consultations are impractical or impossible. For example, geographical, scheduling, and/or other logistical constraints may preclude the patient from meeting directly with the service provider, which may delay or prevent initiation of a medical treatment. Accordingly, the systems, kits, and methods disclosed herein generally are directed to alleviating such difficulties by enabling the service provider to evaluate a medical condition of the patient and/or to develop a treatment plan for the patient without requiring direct and/or in-person interaction with the patient. Hence, utilizing the systems, kits, and/or methods disclosed herein may enable the service provider to diagnose a greater number of patients, and/or patients for whom an in-person screening would be impractical.

As used herein, the term “service provider” may refer to any appropriate medical professional, such as a medical professional in an oral and/or dental field. As examples, the service provider may be a dentist, an orthodontist, an oral surgeon, an endodontic specialist, a periodontist, an otolaryngologist, and/or an otorhinolaryngologist. As used herein, the term “patient” may refer to any appropriate individual with a medical condition to be treated by a medical professional. In some cases (but not all), the patient may be a child, such as a child that is younger than 7 years of age and/or younger than 3 years of age. In such cases, diagnostic communication system 1000 may facilitate the service provider diagnosing a developmental condition of the patient at an early age, thereby reducing a total time and/or expense for treating the condition relative to a case in which the condition is discovered later in life. For other children and/or patients, diagnostic communication system 1000 may enable early and/or periodic diagnosis and/or monitoring of an oral or dental condition. For example, this may enable the condition, including the severity thereof and/or treatment options therefor, to be diagnosed without and/or before requiring the patient to travel to be evaluated and/or treated directly by the service provider. As another example, diagnostic communication system 1000 may effectively enable an individual to obtain a diagnosis of a dental or oral condition from one, or even many, service providers without needing to invest the time and resources for such in-person appointments. Similarly, service providers may utilize diagnostic communication system 1000 to provide diagnoses of oral and/or dental conditions of a greater number of prospective or actual patients due to not needing to expend the time and resources to schedule and perform in-person appointments with such patients.

While the systems and methods described herein generally relate to coordinating information transfer regarding a given (e.g., a particular) patient and a given (e.g., a particular) service provider, it is within the scope of the present disclosure that certain methods and/or steps thereof need not be performed by the given patient and/or the given service provider. As an example, such as when the patient is a minor and/or a child, one or more methods may be performed by and/or directed to a patient proxy that represents and/or otherwise is directly affiliated with the patient, such as a relative of the patient, a parent of the patient, and/or a caretaker of the patient. Additionally or alternatively, one or more methods may be performed by and/or directed to the patient themselves. Accordingly, as used herein, the term “patient representative,” as used to describe one or more individuals acting on behalf of the patient, may be understood as referring to the patient and/or to the patient proxy. As an example, references herein to the patient representative utilizing data collection kit 100 to characterize an oral condition of the patient may refer to an action that is performed by the patient proxy and/or to an action that is performed by the patient themselves. In this manner, each instance of the term “patient representative” herein is intended to refer to one or both of the patient and the patient proxy, and separate instances of the term “patient representative” herein may refer to correspondingly distinct individuals.

Similarly, in some examples, one or more methods may be performed by a service provider proxy that represents and/or otherwise is directly affiliated with the service provider, such as a colleague of the service provider, an assistant to the service provider, and/or a supervisor of the service provider. Additionally or alternatively, one or more methods may be performed by the service provider themselves. Accordingly, as used herein, the term “service provider representative,” as used to describe one or more individuals acting on behalf of the service provider, may be understood as referring to the service provider and/or to the service provider proxy. As an example, references herein to the service provider representative developing a treatment plan to treat an oral condition of the patient may refer to an action that is performed by the service provider proxy and/or to an action that is performed by the service provider themselves. In this manner, each instance of the term “service provider representative” herein is intended to refer to one or both of the service provider and the service provider proxy, and separate instances of the term “service provider representative” herein may refer to correspondingly distinct individuals.

As schematically illustrated in FIG. 1, diagnostic communication system 1000 is configured to coordinate information transfer between a patient electronic device 60 associated with the patient and a service provider electronic device 80 associated with the service provider. More specifically, and as described in more detail herein, diagnostic communication system 1000 is configured to coordinate transfer of an oral condition data set 160 and/or of a treatment plan 170 between the patient representative and the service provider representative via the patient electronic device 60 and the service provider electronic device 80. As discussed in more detail herein, oral condition data set 160 includes data that characterizes an oral condition of the patient, while treatment plan 170 pertains to a proposed plan, procedure, and/or series of procedures to be performed by the service provider to treat the oral condition. As used herein, treatment plan 170 also may be referred to as a proposed treatment plan 170.

Patient electronic device 60 and service provider electronic device 80 each may include and/or be any appropriate device for inputting, transmitting, and/or receiving information, such as oral condition data set 160 and/or treatment plan 170. As examples, each of patient electronic device 60 and service provider electronic device 80 may include and/or be an Internet-connected device, a desktop computer, a laptop computer, a tablet computer, a mobile electronic device, a handheld electronic device, and/or a cellular/smart phone.

In some examples, and as further schematically illustrated in FIG. 1, diagnostic communication system 1000 includes a centralized service 70 that coordinates, enables, and/or facilitates information transfer between patient electronic device 60 and service provider electronic device 80, as described herein. More specifically, in some examples, and as described herein, centralized service 70 is configured to provide data collection kit 100 to the patient, the patient representative, the service provider, and/or the service provider representative. For example, centralized service 70 may be configured to provide data collection kit 100 to the service provider representative such that the service provider representative may distribute data collection kit 100 to one or more patient representatives. In some examples, and as described herein, centralized service 70 is configured to receive oral condition data set 160 from the patient (or patient representative). Additionally or alternatively, in some examples, and as described herein, centralized service 70 is configured to facilitate delivering oral condition data set 160 from the patient (or patient representative) to the service provider (or service provider representative) (such as from patient electronic device 60 to service provider electronic device 80). In some examples, and as described herein, centralized service 70 is configured to facilitate conveying treatment plan 170 from the service provider (or service provider representative) to the patient (or patient representative), such as from service provider electronic device 80 to patient electronic device 60. In some examples, and as discussed in more detail herein, centralized service 70 is configured to perform at least a portion of methods 300, such as to generate at least a portion, and optionally all, of treatment plan 170.

In some examples, one or both of patient electronic device 60 and service provider electronic device 80 is configured to interface with centralized service 70. In some such examples, centralized service 70 includes a computer program (e.g., an executable application) that is configured to be executed on one or both of patient electronic device 60 and service provider electronic device 80. Additionally or alternatively, centralized service 70 may include a computer program (e.g., an executable application) that is configured to be executed on an electronic device (e.g., a computing server) that is spatially removed from each of patient electronic device 60 and service provider electronic device 80. For example, centralized service 70 may be associated with, and/or provided in conjunction with, a subscription service to which the patient and/or the service representative subscribes.

In some examples, and as further schematically illustrated in FIG. 1 and described in more detail herein, centralized service 70 and/or service provider electronic device 80 includes a media resource library 180 that includes, hosts, stores, and/or otherwise provides access to a plurality of diagnostic media resources 182. As examples, and as described in more detail herein in the context of methods 300 of developing the treatment plan, media resource library 180 may include diagnostic media resources 182 in the form of one or more explanatory resources 184, one or more diagnostic reference images 186, one or more target reference images 188, and/or one or more treatment plan media files 190.

Oral condition data set 160 may include and/or be data in any appropriate form and/or quantity, and oral condition data set 160 may pertain to any appropriate condition of the patient. FIG. 2 illustrates an example of a patient’s mouth 10, such as may include one or more structures and/or features characterized by oral condition data set 160. As examples, and with reference to FIG. 2, oral condition data set 160 may include information that pertains to the patient’s mouth 10, the patient’s gums 22, the patient’s tongue 24, the patient’s palate 26, the patient’s airway 28, the patient’s jaw 30, the patient’s maxilla 32, the patient’s mandible 40, and/or the patient’s teeth 50. As used herein, the term “oral,” as used to describe a condition and/or anatomical structure of the patient, may be used to refer to any appropriate structure that defines, that is defined by, that is contained within, or that is otherwise associated with the patient’s mouth 10. Similarly, as used herein, the term “mouth” generally refers to the collection of all anatomical structures formed within and/or adjacent to the patient’s oral cavity, such as the patient’s gums 22, tongue 24, palate 26, airway 28, jaw 30, maxilla 32, mandible 40, and/or teeth 50. In this manner, oral condition data set 160 may include and/or be any appropriate data that characterizes one or more of these anatomical structures, such as orthodontic data, dental data, intraoral data, endodontic data, periodontic data, otolaryngological data, and/or otorhinolaryngological data.

Oral condition data set 160 may include and/or be data in any appropriate form, such as measurement data (e.g., data produced by empirical measurement), numerical data, and/or text data. Additionally or alternatively, and as schematically illustrated in FIG. 1, oral condition data set 160 may include and/or be at least one patient photograph 162 depicting at least a portion of the patient’s mouth 10. In such examples, patient photograph 162 may include and/or be any appropriate photograph, such as a preexisting photograph and/or a photograph that was recorded specifically to be included in oral condition data set 160. As a more specific example, and as schematically illustrated in FIG. 1, patient photograph 162 may be a photograph that was recorded with a camera 62 associated with the patient, such as may be a component and/or an accessory of patient electronic device 60. It is within the scope of the present disclosure that patient photograph 162 additionally or alternatively maybe be referred to as a patient image 162 herein, and such patient image may be recorded with camera 62 and/or may be, or be extracted or produced from, a series of images and/or a video.

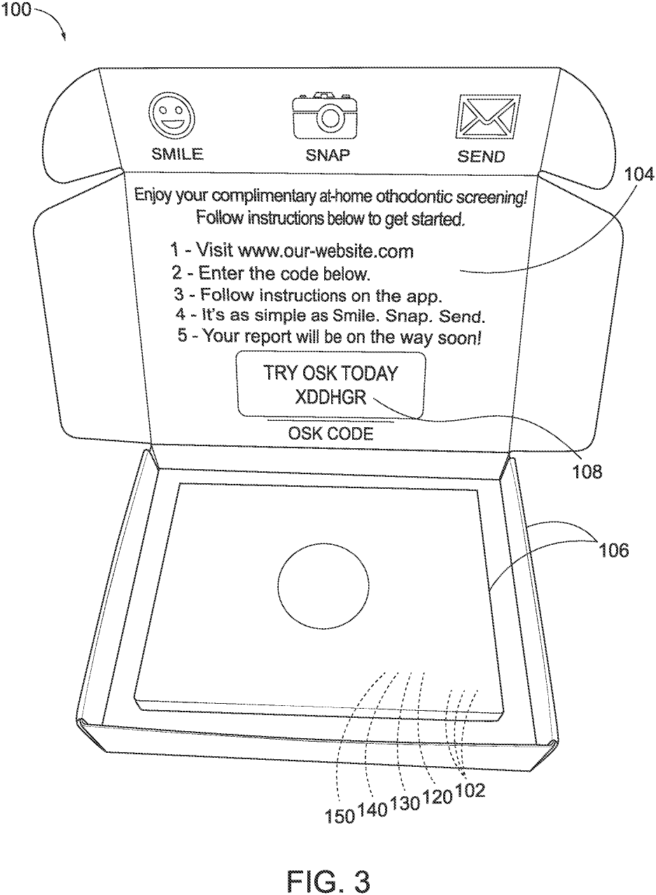

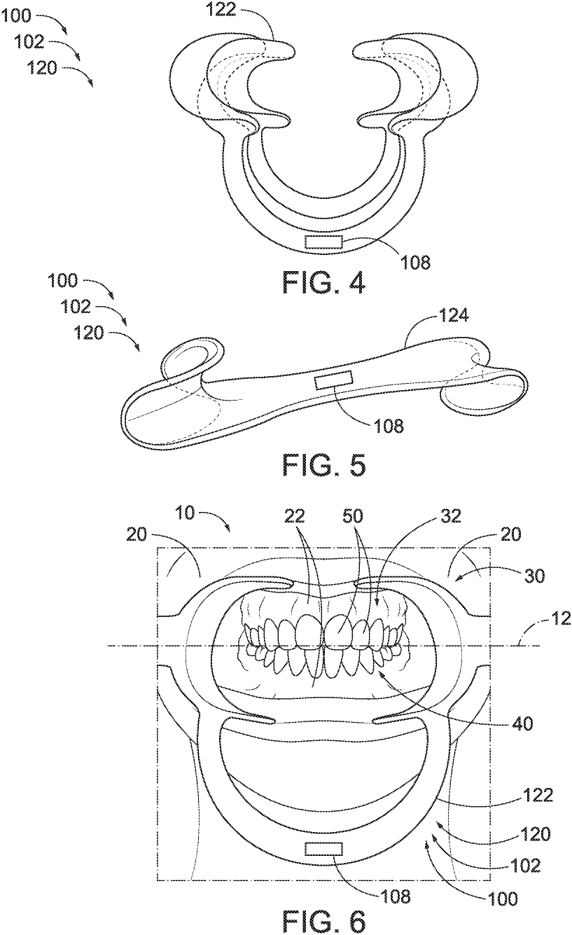

Turning now to FIGS. 3-9, in some examples, oral condition data set 160 is formed, at least in part, using a data collection kit 100, such as may be provided to the patient representative by the service provider representative and/or centralized service 70. FIG. 3 illustrates an example of data collection kit 100, while FIGS. 4-9 illustrate examples of components of data collection kit 100. As shown in FIG. 3, data collection kit 100 includes at least one diagnostic instrument 102 configured to facilitate characterizing at least a portion of the oral condition, instructions 104 for operative use of data collection kit 100, and/or packaging 106 that contains at least diagnostic instrument(s) 102. In some examples, packaging 106 further contains at least a portion of instructions 104. In this manner, each diagnostic instrument 102 is configured to facilitate the generation of oral condition data set 160, while instructions 104 serve to describe a method of generating at least a portion of oral condition data set 160. Thus, providing data collection kit 100 to the patient representative may enable the patient representative to generate oral condition data set 160 without direct action, assistance, and/or intervention by the service provider representative subsequent to the data collection kit 100 being provided to the patient representative.

Diagnostic instruments 102 may include and/or be any appropriate tools and/or devices for facilitating and/or performing measurements of the patient’s mouth 10, such as via direct engagement with at least a portion of the patient’s mouth 10. As examples, and as shown in FIG. 3, diagnostic instruments 102 may include a cheek retractor 120, an overjet gauge 140, and/or a mouth opening gauge 150. When present, cheek retractor 120 is configured to bias the patient’s cheeks 20 apart along an occlusal plane 12 of the patient’s mouth 10 (shown in FIG. 2), such as to provide an unobstructed view of teeth 50. When present, overjet gauge 140 is configured to provide a measurement of a maxillary overjet of the patient’s teeth 50, such as of the patient’s incisor teeth 52 (shown in FIG. 2). When present, mouth opening gauge 150 is configured to provide a measurement of a maximum mouth opening distance 14 of the patient’s mouth 10, such as may be measured between respective incisor teeth 52 of the patient’s maxilla 32 and mandible 40 (shown in FIG. 2) when the patient’s mouth 10 is fully open.

Cheek retractor 120 may include and/or be any appropriate structure for biasing a patient’s cheeks 20 and/or lips apart along occlusal plane 12, or in superior and inferior directions. As an example, and as illustrated in FIG. 4, cheek retractor 120 may include and/or be a one-piece cheek retractor 122 that is configured to engage both cheeks 20 simultaneously to bias (e.g., pull) the cheeks apart. As another example, and as illustrated in FIG. 5, cheek retractor 120 may include and/or be one or more cheek retractor portions 124 (one of which is illustrated in FIG. 5), each of which is configured to engage and bias a respective cheek 20. FIG. 6 illustrates an example of a one-piece cheek retractor 122 during operative use to bias cheeks 20 apart along occlusal plane 12. In some examples, data collection kit 100 includes a plurality of cheek retractors 120 of different sizes such that the patient may select and utilize an appropriately sized and/or most comfortable cheek retractor 120. As used herein, cheek retractor 120 additionally or alternatively may be referred to as a cheek spreader 120. Cheek retractor 120 is not limited to being configured or otherwise used to spread a patient’s cheeks along an occlusal plane. For example, cheek retractor 120 additionally or alternatively may be configured to move a patient’s cheeks and/or lips in a superior (e.g., toward the patient’s eyes) or inferior (e.g., toward the patient’s chin) direction.

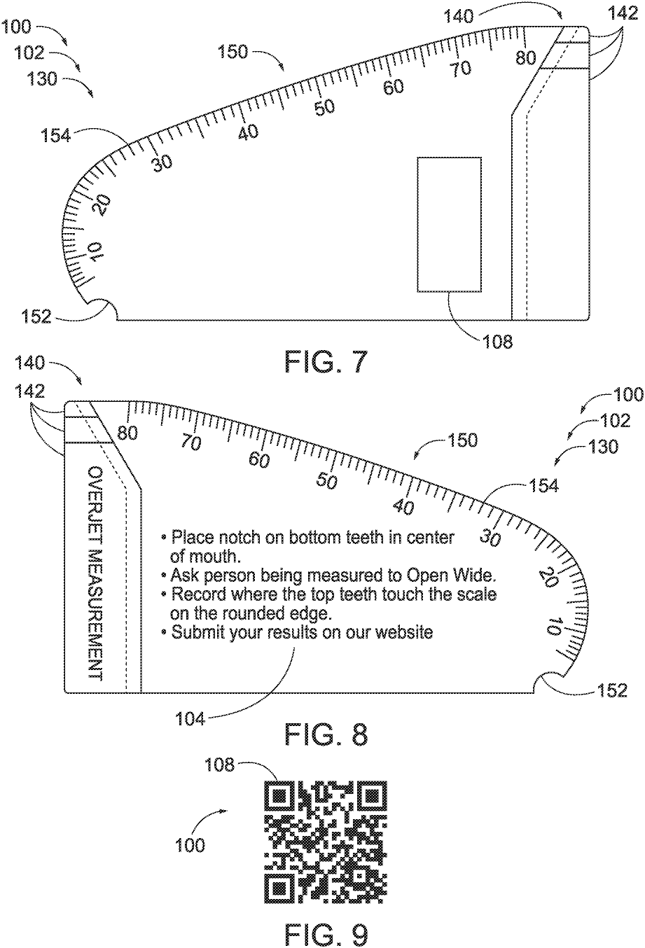

In some examples, data collection kit 100 includes a diagnostic instrument 102 in the form of an integrated gauge tool 130 that includes both of overjet gauge 140 and mouth opening gauge 150. As an example, FIGS. 7-8 illustrate an example of integrated gauge tool 130 that includes overjet gauge 140 and mouth opening gauge 150 integrated on a single card. In such examples, integrated gauge tool 130 may be formed on a plastic card and/or a card that is formed from and/or coated with a waterproof material that is suitable for placement in the patient’s mouth. In some examples, integrated gauge tool 130 is configured such that overjet gauge 140 and mouth opening gauge 150 are initially integrated with one another (e.g., formed on a single card), and integrated gauge tool 130 may be configured to facilitate selective separation of the overjet gauge and the mouth opening gauge from one another by an end user, such as the patient representative. In such examples, integrated gauge tool 130 may include perforations, score lines, cut guides, etc. for facilitating separation of overjet gauge 140 and mouth opening gauge 150 from one another. Data collection kit 100 alternatively may include an overjet gauge 140 that is not integrated with a mouth opening gauge 150 and/or a mouth opening gauge 150 that is not integrated with an overjet gauge 140.

Overjet gauge 140 may be configured to measure the overjet of the patient’s teeth 50 in any appropriate manner. In some examples, overjet gauge 140 is configured to be placed against the patient’s maxilla 32 and/or the patient’s mandible 40 to measure the maxillary overjet between the maxilla and the mandible. As a more specific example, overjet gauge 140 may be configured to be placed against an upper reference tooth 34 of the patient’s maxilla 32 (shown in FIG. 2) and/or a lower reference tooth 42 of the patient’s mandible 40 (shown in FIG. 2) to measure the maxillary overjet between upper reference tooth 34 and lower reference tooth 42, such as while the patient’s jaws 30 are closed. Upper reference tooth 34 and lower reference tooth 42 may be any appropriate teeth, such as respective incisor teeth 52 of the patient’s maxilla 32 and mandible 40.

In some examples, and as illustrated in FIGS. 7-8, overjet gauge 140 may include a plurality of overjet zones 142, such as color-coded overjet zones 142, for classifying, or otherwise determining, the degree of maxillary overjet. In such examples, overjet gauge 140 may be configured to measure the overjet of the patient’s teeth 50 by resting an edge of the overjet gauge against lower reference tooth 42 and recording which overjet zone 142 of overjet gauge 140 is contacted by and/or proximal to upper reference tooth 34.

Mouth opening gauge 150 may be configured to measure maximum opening distance 14 of the patient’s mouth 10 in any appropriate manner. For example, mouth opening gauge 150 may be configured to provide a measurement of mouth opening distance 14 of the patient’s mouth 10 as measured between upper reference tooth 34 of the patient’s maxilla 32 and lower reference tooth 42 of the patient’s mandible 40. In such examples, upper reference tooth 34 and/or lower reference tooth 42 may be the same teeth that are utilized for measurement of the patient’s maxillary overjet with overjet gauge 140, or may refer to a distinct pair of reference teeth.

In some examples, and as illustrated in FIGS. 7-8, mouth opening gauge 150 includes a notch 152 configured to engage upper reference tooth 34 or lower reference tooth 42, and further includes a scale 154 configured to lie adjacent to the other of upper reference tooth 34 and lower reference tooth 42 while notch 152 engages the corresponding tooth 50. In this manner, mouth opening gauge 150 may be configured to quantify a distance between upper reference tooth 34 and lower reference tooth 42 when the patient’s mouth is fully open and when the notch engages the lower reference tooth or the upper reference tooth. As more specific examples, and as shown in FIG. 2, upper reference tooth 34 may be a maxillary central incisor 52, and/or lower reference tooth 42 may be a mandibular central incisor 52.

In some examples, mouth opening gauge 150 further is configured to provide a measurement of a severity of ankyloglossia (also known as tongue-tie) in the patient’s mouth 10. For example, mouth opening gauge 150 may be configured to measure a constrained maximum opening distance of the patient’s mouth 10, such as a maximum distance to which the patient is able to open the patient’s mouth 10 while maintaining the patient’s tongue 24 in contact with a lingual reference point 16 on the roof of the patient’s mouth (shown in FIG. 2). Lingual reference point 16 may be any appropriate location, such as a lingual side of the patient’s mandibular central incisors and/or the point at which the lingual side of the patient’s mandibular central incisors contact the patient’s gums 22.

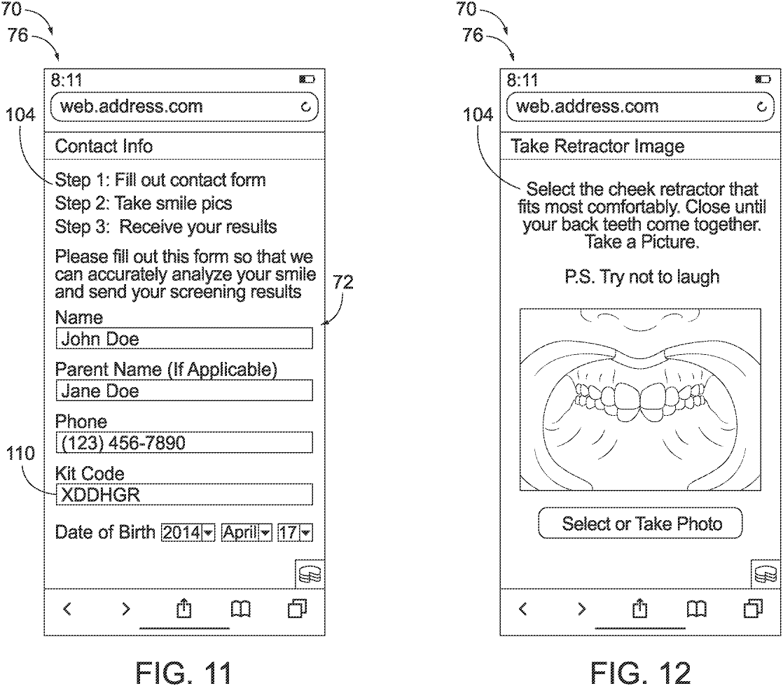

In some examples, and as further shown in FIGS. 3-7, data collection kit 100 further includes a tracking code 108 for at least partially identifying one or more aspects of data collection kit 100. Tracking code 108 may include and/or be any appropriate information, such as an alphanumeric sequence, a word, a sequence of words, a number, a barcode, a URL, a quick response (QR) code, one or more colors, one or more patterns, and/or one or more shapes. As examples, FIG. 3 illustrates an example in which tracking code 108 is an alphanumeric sequence, while FIG. 9 illustrates an example of a tracking code 108 in the form of a QR code. In an example in which data collection kit 100 includes tracking code 108, data collection kit 100 generally is configured such that the resulting oral condition data set 160 includes tracking code information 110 (shown in FIG. 11) relating to tracking code 108, such as a representation of tracking code 108 and/or the tracking code itself. As more specific examples, tracking code information 110 may include and/or be a textual description of tracking code 108 (e.g., a reproduction of the alphanumeric sequence that forms the tracking code, or a textual description of a pattern that forms the tracking code), an image of the tracking code, and/or a photograph of the tracking code.

In an example in which oral condition data set 160 includes tracking code information 110 corresponding to tracking code 108, receipt of tracking code information 110 provides the service provider with additional information regarding the patient, regarding the context in which the patient received data collection kit 100, and/or regarding the source of the data collection kit. As an example, tracking code 108 may be configured to correspond to the identity of the patient to whom data collection kit 100 was provided. As a more specific example, a portion of oral condition data set 160 may include a representation of tracking code 108 (and/or tracking code information 110) in combination with medical information pertaining to the patient, thereby enabling the medical information to be viewed without the viewer also receiving personally identifying information regarding the patient.

In other examples, tracking code 108 may be configured to provide the service provider with non-unique information regarding the patient, such as to identify and/or characterize a grouping of patients and/or of data collection kits 100. As examples, tracking code 108 may be configured to correspond to a date, or date range, on which data collection kit 100 was provided to the patient; a geographical region in which data collection kit 100 was provided to the patient; an identity of a marketing campaign of the service provider and/or the centralized service from which data collection kit 100 was provided to the patient; and/or a unique identifier for identifying the data collection kit among a plurality of data collection kits. Accordingly, in such examples, receiving tracking code information 110 may provide the service provider and/or the centralized service with statistical information regarding the success and/or response rate of a marketing campaign and/or a data collection kit distribution strategy, such as may enable the service provider and/or the centralized service to tailor future deliveries of data collection kit 100 more efficiently. For example, receiving tracking code information 110 in each of a plurality of distinct oral condition data sets 160 corresponding to a respective plurality of data collection kits 100 may enable the service provider and/or the centralized service to distribute additional data collection kits 100 in a manner that is predicted to optimize a likelihood of receiving additional oral condition data sets 160 corresponding to those additional data collection kits 100.

In other examples, tracking code 108 may be configured to correspond to the identity of the service provider to whom oral condition data set 160 is intended to be delivered and/or the identity of the service provider that provided, or authorized the provision of, data collection kit 100 to the patient representative. As an example, data collection kit 100 may be provided to the patient via centralized service 70 (and/or an entity associated with centralized service 70) that represents, and/or that is utilized by, a plurality of distinct service providers. In such examples, tracking code 108 may be configured such that centralized service 70 utilizes tracking code information 110 within oral condition data set 160 to direct the oral condition data set to an appropriate service provider of the plurality of distinct service providers. As a more specific example, a plurality of data collection kits 100 may be distributed to a corresponding plurality of patient representatives that may be served by any of a plurality of distinct service providers, and centralized service 70 may utilize tracking code information 110 to direct each resulting oral condition data set 160 to a service provider that is geographically proximal to the corresponding patient, that is most qualified to diagnose and/or treat the corresponding oral condition, that has engaged the centralized service to obtain and/or provide oral condition data sets 160 to the service provider, that has agreed to receive and/or diagnose oral condition data sets 160, etc.

Data collection kit 100 may include tracking code 108 in any appropriate form and/or manner. As examples, tracking code 108 may be printed on (e.g., printed directly on) one or more diagnostic instruments 102, on instructions 104, and/or on packaging 106. As additional examples, tracking code 108 may be operatively affixed to one or more diagnostic instruments 102, to instructions 104, and/or to packaging 106. In some examples, tracking code 108 is configured to be physically transferred from one component of data collection kit 100 to another component of the data collection kit and/or to oral condition data set 160. As a more specific example, tracking code 108 may be printed on a sticker, a decal, a stamp, and/or a transfer that is configured to be selectively affixed to a component of data collection kit 100, such as diagnostic instrument 102, by an end user, such as the patient representative. As a still more specific example, data collection kit 100 may include tracking code 108 in the form of a sticker that is initially adhered to instructions 104 and/or to packaging 106, and instructions 104 may instruct the patient representative to remove the sticker and replace it upon diagnostic instrument 102. Such instructions 104 further may instruct the patient representative to take a photograph of diagnostic instrument 102 within the patient’s mouth 10 such that the resulting patient photograph 162, and hence the resulting oral condition data set 160, includes tracking code information 110 in the form of a photograph that includes an image of tracking code 108.

Instructions 104 may have any appropriate form and/or content for describing operative use of data collection kit 100 to generate oral condition data set 160. For example, instructions 104 may include instructions for operative use of diagnostic instrument(s) 102 of data collection kit 100 to at least partially characterize the oral condition, as described herein. In some examples, data collection kit 100 includes instructions 104 in printed form, such as may be printed on diagnostic instrument(s) 102, on packaging 106, and/or on a separate instructions sheet or insert that is included within packaging 106. As an example, FIG. 3 illustrates an example in which packaging 106 includes instructions 104 such that the patient representative is presented with the instructions prior to accessing diagnostic instrument(s) 102. As another example, FIG. 8 illustrates an example in which integrated gauge tool 130 includes instructions 104 for operative use thereof. In some examples, instructions 104 may be partial instructions. For example, instructions 104 that are included with data collection kit 100 may represent only an incomplete subset of the full set of instructions 104 that are available to and/or directed to the patient representative. As a more specific example, instructions 104 may include instructions that are configured to be accessed via an Internet connection (such as via centralized service 70). In some such examples, instructions 104 include instructions that are included with data collection kit 100 to direct the patient representative to a Web site that displays the instructions that are configured to be accessed via an Internet connection. In some examples, instructions 104 include instructions for delivering oral condition data set 160 to the service provider, such as via patient electronic device 60 and/or centralized service 70. Instructions 104 additionally or alternatively may include instructions for delivering oral condition data set 160 to centralized service 70.

Packaging 106 may include and/or be any appropriate container and/or storage device for containing elements of data collection kit 100. As examples, packaging 106 may include a container, a resealable container (e.g., a container that is configured to be selectively and repeatedly opened and sealed), a box, and/or a bag. In some examples, packaging 106 is configured to be shipped to the patient representative via a postal service, such as without requiring that packaging 106 be repackaged prior to shipping. In some examples, packaging 106 may include and/or define at least one other element of data collection kit 100. As an example, one or more diagnostic instruments 102 may be at least partially integrated with packaging 106 and may be configured to be selectively removed from the packaging by the patient representative (such as via pre-formed perforations or score lines) prior to utilizing the diagnostic instruments. As another example, at least a portion of instructions 104 may be printed on packaging 106 such that packaging 106 may be described as including instructions 104.

As discussed, diagnostic communication system 1000 generally is configured to support transmission of oral condition data set 160 (such as may be generated by utilizing data collection kit 100) and/or of treatment plan 170 (such as may be based upon oral condition data set 160) between patient electronic device 60 and service provider electronic device 80 directly, or indirectly via centralized service 70. In some examples, and as illustrated in FIGS. 11-14 (described in more detail below), centralized service 70 is configured to generate and/or produce a user interface 76 to be presented to a user (such as the patient representative via patient electronic device 60 and/or service provider representative via service provider electronic device 80). For example, user interface 76 may provide the user with instructions 104 for obtaining and/or use of data collection kit 100, for delivery of oral condition data set 160 to the service provider, for analyzing the oral condition data set by the service provider, and/or for creating, inputting, and/or modifying a treatment plan based on the oral condition data set.

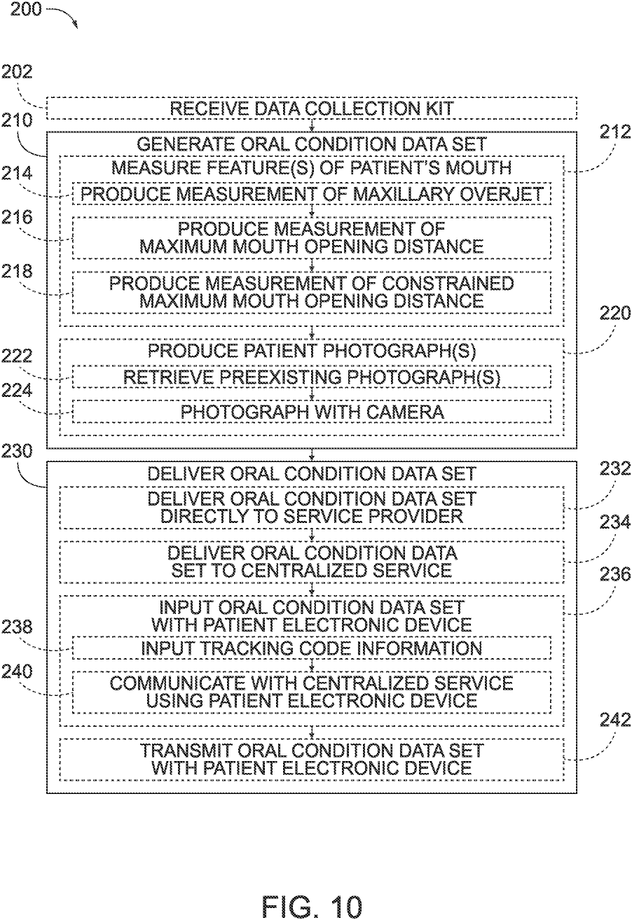

FIG. 10 is a flowchart depicting examples of methods 200, according to the present disclosure, of conveying an oral condition data set (such as oral condition data set 160) from a patient to a service provider and/or a centralized service (such as centralized service 70), as discussed herein. As shown in FIG. 10, methods 200 include generating, at 210, the oral condition data set and delivering, at 230 the oral condition data set. Methods 200 generally are performed, at least in part, by the patient representative, such as the patient and/or the patient proxy, as discussed above.

In some examples, the generating the oral condition data set at 210 is performed, at least in part, by utilizing a data collection kit (such as data collection kit 100). Accordingly, in such examples, method 200 additionally include, prior to the generating the oral condition data set at 210, receiving, at 202, the data collection kit. The receiving the data collection kit at 202 may include obtaining the data collection kit in any appropriate manner, such as by receiving the data collection kit directly from the service provider and/or the centralized service.

The generating the oral condition data set at 210 may include any appropriate steps, such as may be performed in accordance with instructions (such as instructions 104) that are included with the data collection kit. In some examples, and as shown in FIG. 10, the generating the oral condition data set includes measuring, at 212, one or more features of the patient’s mouth, such as by utilizing one or more diagnostic instruments (such as diagnostic instrument(s) 102) that are included with the data collection kit. In some such examples, and as described herein, the measuring the one or more features of the patient’s mouth at 212 includes utilizing the data collection kit such that at least a portion of the diagnostic instrument(s) engages at least a portion of the patient’s mouth. As a more specific example, and as shown in FIG. 10, the measuring the feature(s) of the patient’s mouth at 212 may include producing, at 214, a measurement of a maxillary overjet of the patient’s teeth such that the oral condition data set includes the measurement of the maxillary overjet.

In some examples, the producing the measurement of the maxillary overjet at 214 is performed, at least in part, by utilizing an overjet gauge (such as overjet gauge 140) as described herein. As a more specific example, the producing the measurement of the maxillary overjet at 214 may include utilizing an overjet gauge that includes a plurality of overjet zones (such as overjet zones 142), and the producing the measurement of the maxillary overjet at 214 may include resting an edge of the overjet gauge against a tooth of the patient’s mandible (such as lower reference tooth 42 and/or another tooth 50 of mandible 40) and identifying which overjet zone of the plurality of overjet zones is proximal a tooth of the patient’s maxilla (such as upper reference tooth 34 and/or another tooth 50 of maxilla 32).

In some examples, and as shown in FIG. 10, the measuring the feature(s) of the patient’s mouth may include producing, at 216, a measurement of a maximum opening distance of the patient’s mouth (such as maximum mouth opening distance 14) such that the oral condition data set includes the measurement of the maximum mouth opening distance. In some such examples, the maximum mouth opening distance is measured between a lower reference tooth of the patient’s mandible (such as lower reference tooth 42 of mandible 40) and an upper reference tooth of the patient’s maxilla (such as upper reference tooth 34 of maxilla 32). As a more specific example, the lower reference tooth may be a mandibular central incisor, and the upper reference tooth may be a maxillary central incisor. In an example in which the measuring the feature(s) of the patient’s mouth at 212 includes both the producing the measurement of the maxillary overjet at 214 and the producing the measurement of the maximum mouth opening distance at 216, each measurement may utilize the same upper reference tooth and/or the same lower reference tooth, or the measurements may utilize two different upper reference teeth and/or two different lower reference teeth.

In some examples, the producing the measurement of the maximum mouth opening distance at 216 is performed, at least in part, by utilizing a mouth opening gauge (such as mouth opening gauge 150), as described herein. As a more specific example, the producing the measurement of the maximum mouth opening distance at 216 may include engaging the lower reference tooth or the upper reference tooth with a notch (such as notch 152) of the mouth opening gauge and engaging the other of the lower reference tooth and the upper reference tooth with a scale (such as scale 154) of the mouth opening gauge. In such examples, the producing the measurement of the maximum mouth opening distance at 216 subsequently includes determining a position, relative to the scale, of the reference tooth that engages the scale while the patient’s mouth is fully open. Stated differently, in such examples, the measurement of the mouth opening distance corresponds to the location of the reference tooth that engages the scale relative to the scale, as specified and/or quantified by the scale.

In some examples, and as shown in FIG. 10, the measuring the feature(s) of the patient’s mouth at 212 additionally or alternatively may include producing, at 218, a measurement of a constrained maximum mouth opening distance of the patient’s mouth such that the oral condition data set includes the measurement of the constrained maximum mouth opening distance. In such examples, the constrained maximum mouth opening distance is measured between a lower reference tooth of the patient’s mandible (such as lower reference tooth 42 of mandible 40) and an upper reference tooth of the patient’s maxilla (such as upper reference tooth 34 of maxilla 32). More specifically, the producing the measurement of the constrained maximum mouth opening distance at 218 includes measuring the constrained maximum mouth opening distance between the lower reference tooth and the upper reference tooth while the patient maintains the patient’s tongue (such as tongue 24) in contact with a lingual reference point (such as lingual reference point 16) on the roof of the patient’s mouth (such as on the patient’s palate 26 and/or at a location proximal to the patient’s gums 22). As a more specific example, the lingual reference point may be a lingual side of the patient’s mandibular central incisors. In such examples, the lower and upper reference teeth may be the same reference teeth or different reference teeth than are utilized in the producing the measurement of the maxillary overjet at 214 and/or the producing the measurement of the maximum mouth opening distance at 216.

In some examples, the producing the measurement of the constrained maximum mouth opening distance at 218 is performed, at least in part, by utilizing the mouth opening gauge as described herein. As a more specific example, the producing the measurement of the constrained maximum mouth opening distance at 218 may include engaging the lower reference tooth or the upper reference tooth with the notch of the mouth opening gauge and engaging the other of the lower reference tooth and the upper reference tooth with the scale of the mouth opening gauge. In such examples, the producing the measurement of the constrained maximum mouth opening distance at 218 subsequently includes determining a position, relative to the scale, of the reference tooth that engages the scale while the patient’s mouth is fully open and while the patient’s tongue is maintained in contact with the lingual reference point. Stated differently, in such examples, the measurement of the constrained mouth opening distance corresponds to the location of the reference tooth that engages the scale relative to the scale, as specified and/or quantified by the scale.

In some examples, and as shown in FIG. 10, the generating the oral condition data set at 210 includes producing, at 220, one or more patient photographs (such as patient photograph(s) 162) that represent at least a portion of the patient’s mouth such that the oral condition data set includes the one or more patient photographs. In such examples, the producing the patient photograph(s) at 220 may be performed in any appropriate manner, such as by producing new and/or preexisting photographs. For example, and as shown in FIG. 10, the producing the patient photograph(s) at 220 may include retrieving, at 222, one or more preexisting photographs that represent at least a portion of the patient’s mouth, such as photographs that were owned and/or accessible by the patient representative prior to receipt and/or utilization of the data collection kit. Additionally or alternatively, and as shown in FIG. 10, the producing the patient photograph(s) at 220 may include photographing, at 224, with a camera that is associated with the patient (such as camera 62). As a more specific example, the patient electronic device may include and/or be a cellular/smart phone, tablet, or personal computing device that includes the camera, such that the photographing at 224 is performed with the patient electronic device. In such examples, the photographing at 224 may include photographing any appropriate portion of the patient’s mouth, such as an underside of the patient’s tongue, the patient’s airway (such as airway 28), the patient’s teeth with the patient’s jaw closed, and/or the patient’s teeth with the patient’s jaw open. In some examples, the photographing at 224 includes photographing the patient’s mouth during operative use of one or more diagnostic instruments of the data collection kit. As an example, the data collection kit may include a cheek retractor (such as cheek retractor 120) to facilitate producing an unobstructed image of the patient’s teeth, and the photographing at 224 may include photographing at least a portion of the patient’s mouth while biasing the patient’s cheeks and/or lips apart along an occlusal plane (such as occlusal plane 12) of the patient’s mouth, in a superior and inferior directions with the cheek retractor. However, this is not required, and it is additionally within the scope of the present disclosure that the photographing at 224 additionally or alternatively may include photographing the patient’s mouth without utilizing a diagnostic instrument of the data collection kit. As an example, the photographing at 224 may include photographing while biasing at least one of the patient’s cheeks and/or lips along the occlusal plane, in a superior direction, and/or in an inferior direction with fingers and/or with common household items.

In some examples, the photographing at 224 includes photographing such that at least a portion of a tracking code (such as tracking code 108) is visible in the resulting patient photograph. As an example, one or more diagnostic instruments of the data collection kit may include the tracking code, such that photographing the patient’s mouth during operative use of the diagnostic instrument includes photographing the tracking code as well as the patient’s mouth. In this manner, the oral condition data set may include the tracking code information (such as tracking code information 110) corresponding to the tracking code in conjunction with the producing the patient photograph(s) at 220 without requiring additional action of the patient representative.

The delivering the oral condition data set at 230 may include delivering in any appropriate manner, such as may be performed in accordance with instructions (such as instructions 104) that are included with the data collection kit. The delivering the oral condition data set at 230 also may include delivering the oral condition data set to any appropriate direct (e.g., initial and/or immediate) recipient such that the oral condition data set reaches the service provider. As an example, and as shown in FIG. 10, the delivering the oral condition data set at 230 may include delivering, at 232, at least a portion of the oral condition data set directly to the service provider. In some such examples, the delivering the oral condition data set directly to the service provider at 232 includes physically bringing at least a portion of the oral condition data set to the service provider, such as by bringing the oral condition data set to an office of the service provider in person. In other examples, the delivering the oral condition data set directly to the service provider at 232 may include sending, transmitting, and/or conveying at least a portion of the oral condition data set directly to the service provider, such as via electronic mail, a text message, a telephone call, and/or a postal service. Additionally or alternatively, and as further shown in FIG. 10, the delivering the oral condition data set at 230 may include delivering, at 234, at least a portion of the oral condition data set via a centralized service (such as centralized service 70).

In some examples, the delivering the oral condition data set at 230 includes utilizing a patient electronic device associated with the patient (such as patient electronic device 60) to input and/or transmit the oral condition data set. For example, and as shown in FIG. 10, the delivering the oral condition data set at 230 may include inputting, at 236, the oral condition data set with the patient electronic device and subsequently transmitting, at 242, the oral condition data set with the patient electronic device, such as to the service provider and/or to the centralized service.

The inputting the oral condition data set at 236 may include inputting any suitable portion, and optionally all, of the oral condition data set. As an example, the oral condition data set may include tracking code information (such as tracking code information 110) corresponding to a tracking code (such as tracking code 108) that was provided with the data collection kit. In such examples, and as shown in FIG. 10, the inputting the oral condition data set at 236 may include inputting, at 238, the tracking code information into the patient electronic device. The inputting the tracking code information at 238 may be performed in any appropriate manner, such as may correspond to the form of the tracking code. As an example, the tracking code may include an alphanumeric sequence, and the inputting the tracking code information at 238 may include manually inputting the tracking code information, such as by reproducing the alphanumeric string with the patient electronic device (e.g., with a keyboard). As another example, the oral condition data set may include a patient photograph that includes the tracking code information in the form of an image of at least a portion of the tracking code, and the inputting the tracking code information at 238 may include uploading the patient photograph that includes the tracking code information.

As discussed, the delivering the oral condition data set at 230 may include the delivering the oral condition data set to the centralized service at 234. In such examples, and as shown in FIG. 10, the inputting the oral condition data set at 236 may include communicating, at 240, with the centralized service using the patient electronic device. As examples, the communicating with the centralized service at 240 may include accessing the centralized service, delivering a communication and/or the oral condition data set to the centralized service, and/or receiving a communication and/or a prompt for the oral condition data set from the centralized service. In some examples, the delivering the oral condition data set to the centralized service at 234 and/or the accessing the centralized service at 240 may be performed, at least in part, via an Internet connection. For example, the communicating with the centralized service at 240 may include utilizing the patient electronic device to access a Web page associated with the centralized service and/or a mobile application associated with the centralized service (such as an application that communicates with the centralized service via an Internet connection).

FIGS. 11-14 illustrate examples of user interfaces 76 associated with and/or provided by centralized service 70, such as may be displayed to the patient representative via patient electronic device 60. In particular, FIGS. 11-14 illustrate examples of Web sites associated with centralized service 70, such as may direct and/or facilitate execution of methods 200 by the patient representative. For example, each of FIGS. 11-13 illustrates an example of user interface 76 that provides the user with instructions 104 for performing one or more aspects of methods 200.

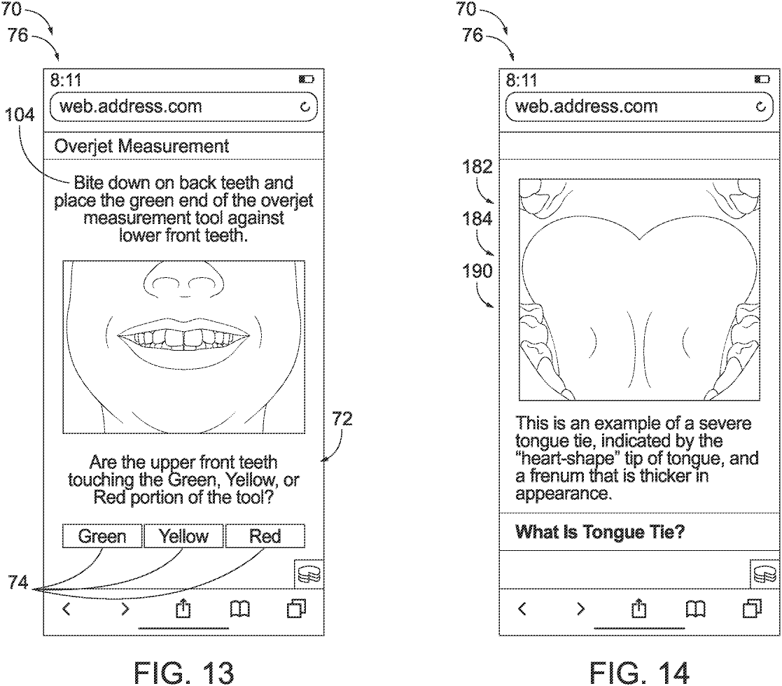

FIGS. 11 and 13 additionally illustrate examples in which user interface 76 includes an input module 72 for receiving patient information and/or components of oral condition data set 160. More specifically, FIG. 11 illustrates an example of user interface 76 in which input module 72 enables the patient representative to input information regarding the patient, as well as tracking code information 110 corresponding to a tracking code provided by the data collection kit, such as tracking code 108 of data collection kit 100 illustrated in FIG. 3. In such examples, the inputting the oral condition data set with the patient electronic device at 236 may include inputting data (such as text data and/or numerical data) via a keyboard of and/or associated with the patient electronic device. As a more specific example, the inputting the oral condition data set with the patient electronic device at 236 may include entering, with the keyboard, a numerical value of a maximum mouth opening distance as obtained with a mouth opening gauge (such as mouth opening gauge 150).

FIG. 13 illustrates an example of user interface 76 in which input module 72 includes a plurality of selectable options 74 corresponding to aspects of oral condition data set 160. Specifically, FIG. 13 illustrates an example in which options 74 correspond to overjet zones that may be measured by an overjet gauge, such as overjet zones 142 of overjet gauge 140 of FIGS. 7-8. In this manner, the inputting the oral condition data set with the patient electronic device at 236 may include entering at least a portion of the oral condition data set into an input module (such as input module 72 of either of FIGS. 11 and 13) that is generated by the centralized service. Similarly, in some such examples, the input module may be configured to permit the user to select at least one option of a plurality of options (such as options 74 of FIG. 13), such that the inputting the oral condition data set at 236 includes entering at least a portion of the oral condition data set into the input module by selecting at least one option of the plurality of options. Additionally, FIG. 12 illustrates an example of user interface 76 that includes instructions 104 for providing a patient photograph (such as patient photograph 162) to centralized service 70. Accordingly, FIG. 12 may be described as illustrating an example of user interface 76 to be generated and/or utilized in an example in which the inputting the oral condition data set at 236 includes uploading one or more patient photographs to the centralized service with the patient electronic device.

In some examples, such as in the example shown in FIG. 14, user interface 76 may be at least substantially informational, such as a Web page that does not instruct the user to perform a task or to enter an input. More specifically, FIG. 14 illustrates an example of user interface 76 that presents an example of a treatment plan media file 190, as discussed in more detail below.

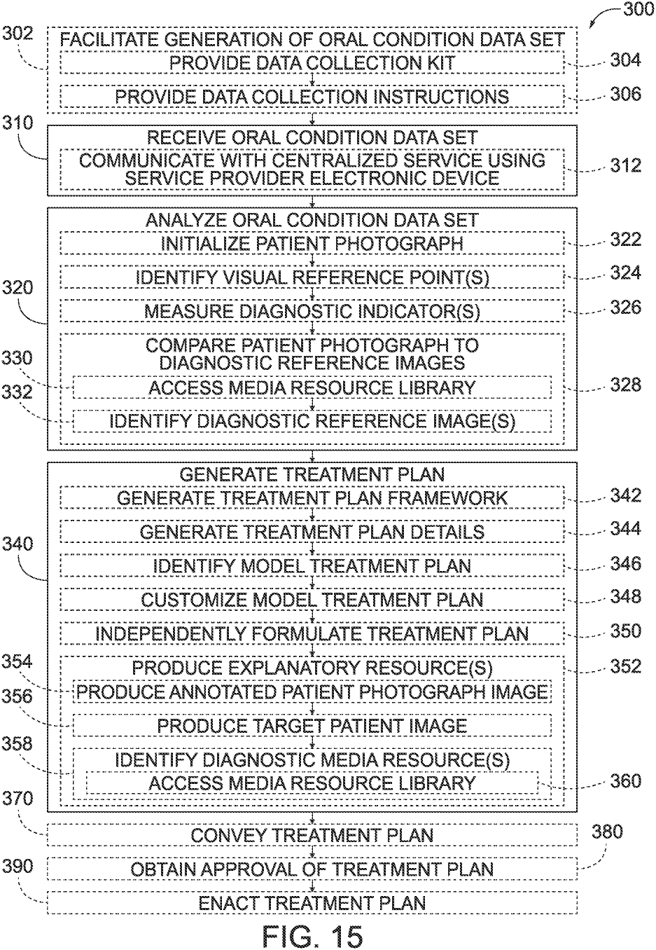

FIG. 15 is a flowchart depicting examples of methods 300, according to the present disclosure, of developing a treatment plan (such as treatment plan 170) to be performed by a service provider to treat an oral condition of a patient, as discussed herein. As shown in FIG. 15, methods 300 include receiving, at 310, an oral condition data set (such as oral condition data set 160) that characterizes the oral condition and analyzing, at 320, the oral condition data set. Methods 300 further include generating, at 340, the treatment plan based, at least in part, on the analyzing the oral condition data set at 320. In some examples, the receiving the oral condition data set at 310 and the analyzing the oral condition data set at 320 pertain to the same oral condition data set, or at least a portion (e.g., a particular portion) thereof, that is generated via the generating the oral condition data set at 210 and/or that is delivered via the delivering the oral condition data set at 230. Stated differently, in some examples, methods 200 and methods 300 as discussed herein pertain to the same patient, the same oral condition of the patient, the same oral condition data set, and/or the same treatment plan, as viewed from the patient side and the service provider side, respectively. In this manner, methods 300 may be performed responsive to any and/or all of methods 200, or may be performed at least partially independent of methods 200. Methods 300 (or a portion thereof) may be performed by any appropriate entity, such as the service provider representative (i.e., the service provider and/or the service provider proxy) and/or the centralized service, as described herein.

The receiving the oral condition data set at 310 may include receiving the oral condition data set via any appropriate process and/or device, such as via a service provider electronic device associated with the service provider (such as service provider electronic device 80). As an example, the centralized service may be configured to host, store, and/or route at least a portion of the oral condition data set. In such examples, and as shown in FIG. 15, the receiving the oral condition data set at 310 may include communicating, at 312, with the centralized service using the service provider electronic device. As examples, the communicating with the centralized service at 312 may include accessing the centralized service, delivering a communication and/or a request for the oral condition data to the centralized service, and/or receiving a communication and/or the oral condition data set for information from the centralized service. As a more specific example, the communicating with the centralized service at 312 may include communicating with the centralized service via an Internet connection, and/or may include accessing a program and/or aspect of the centralized service that is stored and/or executed on the service provider electronic device. In such examples, the receiving the oral condition data set at 310 may be described as being performed by the service provider representative. Additionally or alternatively, the receiving the oral data set at 310 may be described as being performed at least partially by the centralized service, such as in an example in which the delivering the oral condition data set at 230 includes the delivering the oral condition data set to the centralized service at 234.

In some examples, and as shown in FIG. 15, method 300 further includes, prior to the receiving the oral condition data set at 310, facilitating, at 302, generation of the oral condition data set. Stated differently, in such examples, method 300 includes one or more steps for enabling and/or facilitating the performance of the generating the oral condition data set at 210, such as by the patient representative. As more specific examples, and as shown in FIG. 15, the facilitating generation of the oral condition data set at 302 may include providing, at 304, a data collection kit (such as data collection kit 100) to the patient representative and/or providing, at 306, data collection instructions (such as instructions 104) to the patient representative. The providing the data collection kit to the patent representative at 304 may include providing the data collection kit directly to the patient representative, such as by delivering the data collection kit via a postal service and/or providing the data collection kit to a patient representative that visits an office of the service provider. Additionally or alternatively, the providing the data collection kit to the patient representative at 304 may include indirectly providing the data collection kit to the patient representative and/or making the data collection kit available to the patient representative. For example, the data collection kit may be one of a plurality of data collection kits that are distributed to a plurality of patient representatives, such as at a public event and/or by the centralized service (or an entity associated with the centralized service), without requiring that the patient representative and/or the service provider have prior knowledge of one another. As additional examples, at least a portion of the data collection kit (such as one or more diagnostic instruments 102 and/or instructions 104) may be printed by the patient representative, such as after the kit is received by and/or sent to the patient representative via email or digital message or downloaded or accessed by the patient representative from a Web site or other Internet-accessible domain. The providing the data collection instructions at 306 may include providing instructions (such as instructions 104) for performing any appropriate aspect of methods 200, as described herein.

In some examples, the providing the data collection kit at 304 is based, at least in part, on tracking code information, such as prior tracking code information that was included in an oral condition data set that previously was provided by a prior patient of the service provider. For example, such prior tracking code information may correspond to the tracking code of a different data collection kit that previously had been distributed by, or on behalf of, the service provider. In such examples, the tracking code information (and/or the service provider’s prior receipt thereof) may provide the service provider with information regarding a prior response rate of oral condition data sets received relative to the number of data collection kits distributed. Such information thus may enable the service provider and/or the centralized service to generate a strategy for optimizing the distribution of the data collection kits, such as to maximize a response rate. Accordingly, the providing the data collection kit at 304 may include distributing the data collection kits in a manner that is predicted to optimize and/or maximize a response rate. As an example, the response rate may correspond to a ratio of the number of unique data collection kits that are distributed (e.g., to a group or within a geographical region) to the number of corresponding oral condition data sets that subsequently are received by the service provider.

The analyzing the oral condition data set at 320 may be performed by any appropriate entity or entities, and may include any appropriate analyses. As examples, the analyzing the oral condition data set at 320 may be performed, at least in part, by the service provider representative, and/or may be performed, at least in part, by an automated process, such as an automated process of the centralized service. Stated differently, it is within the scope of the present disclosure that the analyzing the oral condition data set at 320 may be performed at least partially automatically.

The analyzing the oral condition data set at 320 may include calculating and/or analyzing any appropriate metrics corresponding to the oral condition data set. For example, the analyzing the oral condition data set at 320 may include analyzing data that is produced by one or more diagnostic instruments (such as diagnostic instrument(s) 102) that are provided to the patient representative, such as with the data collection kit. As a more specific example, the analyzing the oral condition data set at 320 may include analyzing a measurement of a maxillary overjet of the patient’s jaw, such as may be produced using an overjet gauge (such as overjet gauge 140) and/or via the producing the measurement of the maxillary overjet at 214. As another example, the analyzing the oral condition data set at 320 may include analyzing a measurement of a maximum mouth opening distance and/or a constrained maximum mouth opening distance of the patient’s mouth, such as may be produced using a mouth opening gauge (such as mouth opening gauge 150) and/or via the producing the measurement of the maximum mouth opening distance at 216 and/or via the producing the measurement of the constrained maximum mouth opening distance at 218.

In some examples, the analyzing the oral condition data set at 320 includes analyzing graphical and/or image data. As an example, the oral condition data set may include one or more patient photographs (such as patient photograph(s) 162) depicting at least a portion of the patient’s mouth, and the analyzing the oral condition data set at 320 may include analyzing at least one of the patient photographs. In such examples, and as shown in FIG. 15, the analyzing the oral condition data set at 320 may include initializing, at 322, the patient photograph, such as by cropping the patient photograph to a region of interest, rescaling one or more dimensions of the patient photograph to a standardized dimension, and/or rotating the patient photograph to a standardized orientation. As more specific examples, the initializing the patient photograph at 322 may include manipulating the patient photograph such that only the patient’s mouth is shown; such that an occlusal plane (such as occlusal plane 12) of the patient’s mouth is at least substantially horizontal, and/or such that an aspect ratio of the patient photograph is a predetermined aspect ratio. Such initialization steps may be beneficial and/or necessary for subsequent analysis of the patient photograph by an automated process and/or by the centralized service, and/or may facilitate the service provider representative interpreting the patient photograph. In some examples, the initializing the patient photograph at 322 is performed at least substantially automatically (such as by centralized service 70), such as prior to displaying the patient photograph to the service provider.

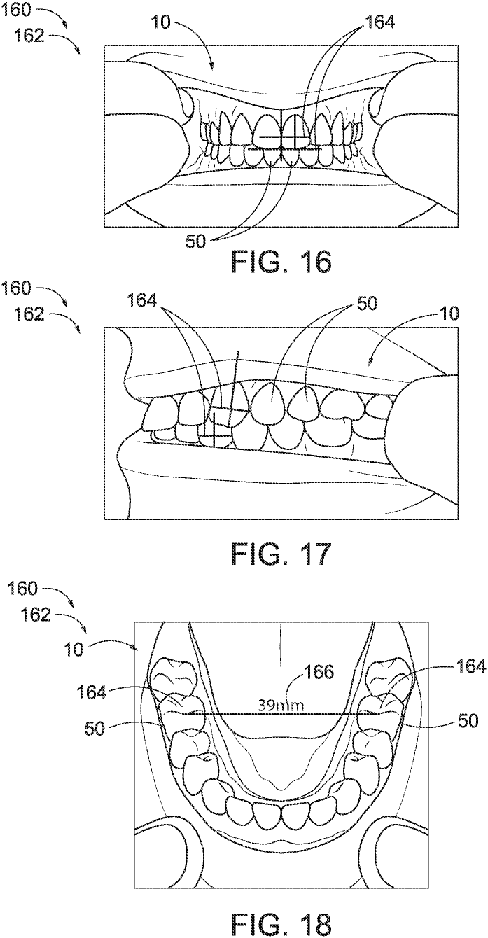

In some examples, and as shown in FIG. 15, the analyzing the oral condition data set includes identifying, at 324, one or more visual reference points of the patient photograph and/or measuring, at 326, one or more diagnostic indicators from the patient photograph, such as may be measured with reference to the visual reference points. As more specific examples, FIGS. 16-18 illustrate examples of patient photographs 162 of oral condition data set 160 depicting a portion of a patient’s mouth 10 in which a plurality of visual reference points 164 have been identified (such as via the identifying the one or more visual reference points at 324). FIG. 18 further illustrates an example in which visual reference points 164 are utilized to measure a diagnostic indicator 166 characterizing the patient’s mouth, such as may be produced via the measuring the one or more diagnostic indicators at 326.

In some examples, and as shown in FIG. 15, the analyzing the oral condition data set at 320 includes comparing, at 328, a patient photograph to one or more diagnostic reference images (such as diagnostic reference images 186), such as to evaluate the oral condition qualitatively and/or relative to known oral conditions. As a more specific example, and as shown in FIG. 15, the comparing the patient photograph to the diagnostic reference image(s) at 328 may include accessing, at 330, a media resource library (such as media resource library 180) that includes a plurality of diagnostic reference images and identifying, at 332, one or more diagnostic reference images of the plurality of diagnostic reference images based upon a similarity to the patient photograph. As a more specific example, the plurality of diagnostic reference images 186 may correspond to a respective plurality of oral conditions (and/or instances, types, forms, severities, etc. of such oral conditions), and the identifying the one or more diagnostic reference images at 332 may include identifying a diagnostic reference image that is most visually similar to the patient photograph. In this manner, the comparing the patient photograph to the diagnostic reference image(s) at 328 may correspond to identifying and/or evaluating the oral condition of the patient via direct comparison with preexisting images corresponding to known oral conditions. As a more specific example, the patient photograph may include an image of the patient’s airway, and the comparing the patient photograph to the diagnostic reference image(s) at 328 may include comparing the patient photograph to images representing Mallampati classifications in order to evaluate a Mallampati score corresponding to the patient. The diagnostic reference images may include and/or be any appropriate images, such as photographs, photographs of prior patients of the service provider, illustrations, animations, etc. The diagnostic reference images may include images associated with and/or provided by the service provider, and/or may include images associated with and/or provided by the centralized service.

The generating the treatment plan at 340 may be performed in any appropriate manner and by any appropriate entity, and may yield a treatment plan of any appropriate form. The treatment plan produced by the generating the treatment plan at 340 may be directed to treating, alleviating, and/or curing any of a plurality of oral conditions, examples of which include cross bite, dental crowding, narrow arches, underbite, overbite, dead teeth, missing teeth, early tooth loss, asymmetrical dental growth, dental midline discrepancies, ankyloglossia, and/or dental malocclusion. The treatment plan may include one or more proposed medical operations for treating the oral condition, a proposed itinerary (e.g., a timeline and/or a schedule) for performing the proposed medical operations, and/or an anticipated expense for performing the proposed medical operations.

In some examples, and as described herein, the generating the treatment plan at 340 is performed, at least in part, by the service provider representative. As a more specific example, the analyzing the oral condition data set at 320 may be performed, at least in part, by an automated process (such as by an automated process of the centralized service), and the generating the treatment plan at 340 may be performed by the service provider representative based on the analysis performed by the automated process. Stated differently, the analyzing the oral condition data set at 320 may include automatically screening, filtering, sorting, and/or otherwise initially processing the oral condition data set to facilitate and/or simplify the generating the treatment plan at 340 as performed by the service provider representative. Additionally or alternatively, and as further described herein, the generating the treatment plan at 340 may be performed, at least in part, by an automated process, such as an automated process of the centralized service.

In some examples, and as shown in FIG. 15, the generating the treatment plan at 340 includes generating, at 342, a treatment plan framework that includes one or more general treatment plan steps and subsequently generating, at 344, treatment plan details. In such examples, the treatment plan details include one or more specific substeps for accomplishing, performing, and/or executing the general treatment plan steps. In this manner, the treatment plan framework may be described as representing an outline and/or summary of the treatment plan, such as may represent high-level components and/or concepts of the treatment plan, while the treatment plan details may be described as representing the specific details, actions, and/or characteristics corresponding to the elements of the treatment plan framework.

In some examples, the generating the treatment plan framework at 342 and the generating the treatment plan details at 344 are performed by separate entities. FIG. 1 schematically illustrates examples of such methods. In particular, FIG. 1 schematically illustrates the selective communication of a treatment plan framework 172 between centralized service 70 and service provider electronic device 80. As an example, the generating the treatment plan framework (such as treatment plan framework 172 of FIG. 1) at 342 may be performed, at least in part, by the service provider representative, and the generating the treatment plan details at 344 may be performed, at least in part, by the centralized service. For example, the service provider representative may decide upon the basic steps and/or goals of the treatment plan, and the centralized service subsequently may generate corresponding treatment plan details, thereby reducing an expenditure of time on the part of the service provider representative. Accordingly, in such examples, and as schematically illustrated in FIG. 1, the generating the treatment plan at 340 may include transmitting treatment plan framework 172 from service provider electronic device 80 to centralized service 70. In an example in which the centralized service operates on the service provider electronic device, the generating the treatment plan at 340 may include entering and/or uploading the treatment plan framework to the centralized service with the service provider electronic device.

As another example, the generating the treatment plan framework at 342 may be performed, at least in part, by the centralized service, and the generating the treatment plan details at 344 may be performed, at least in part, by the service provider representative. For example, the centralized service may perform at least a portion of the analyzing the oral condition data set at 320, and the centralized service subsequently may generate at least a portion of the treatment plan framework based upon the oral condition data set. In such examples, the centralized service may generate at least a portion of the treatment plan framework based upon prior examples of oral condition data sets and corresponding treatment plans, and the service provider representative subsequently may refine the treatment plan framework and/or provide treatment plan details to more fully flesh out the treatment plan. Accordingly, in such examples, and as schematically illustrated in FIG. 1, the generating the treatment plan at 340 may include transmitting treatment plan framework 172 from centralized service 70 to service provider electronic device 80. In an example in which the centralized service operates on the service provider electronic device, the generating the treatment plan at 340 may include displaying and/or otherwise making available the treatment plan framework upon the provider electronic device.

The generating the treatment plan at 340 may include formulating a new treatment plan that is specific to the patient, and/or may include utilizing one or more aspects of a preexisting treatment plan. For example, and as shown in FIG. 15, the generating the treatment plan at 340 may include identifying, at 346, a model treatment plan from among a plurality of preexisting treatment plans. In such examples, the preexisting treatment plans may include treatment plans previously utilized by the service provider, treatment plans provided by and/or associated with the centralized service, and/or treatment plans that otherwise are known to the medical community. In some such examples, and as shown in FIG. 15, the generating the treatment plan at 340 includes customizing, at 348, the model treatment plan for the oral condition of the patient. For example, the identifying the model treatment plan at 346 may include identifying a model treatment plan that is approximately, or most closely, suitable and/or adapted for treating the oral condition of the patient, and the customizing the model treatment plan at 348 may include adjusting, supplementing, and/or otherwise altering the model treatment plan to be more precisely adapted to the oral condition of the patient. Additionally or alternatively, and as shown in FIG. 15, the generating the treatment plan at 340 may include independently formulating, at 350, at least a portion of the treatment plan. For example, the independently formulating the treatment plan at 350 may include producing at least a portion of the treatment plan that is not immediately based upon a preexisting treatment plan. In all such examples, the identifying the model treatment plan at 346, the customizing the model treatment plan at 348, and/or the independently formulating the treatment plan at 350 may pertain to any appropriate aspect and/or component of the treatment plan, such as the treatment plan framework and/or the treatment plan details.

In some examples, at least a portion of the treatment plan is configured to be transmitted to the patient representative. Accordingly, in some examples, and as shown in FIG. 15, the generating the treatment plan at 340 includes producing, at 352, one or more explanatory resources (such as explanatory resources 184 schematically illustrated in FIG. 1) for describing at least a portion of the treatment plan to the patient representative. In some such examples, the treatment plan includes the explanatory resources. As described herein, the producing the one or more explanatory resources at 352 may include identifying preexisting explanatory resources, and/or may include creating and/or generating the explanatory resources.

Each explanatory resource may include and/or be any appropriate resource for conveying at least a portion of the treatment plan to the patient representative. For example, and as shown in FIG. 15, the producing the one or more explanatory resources at 352 may include producing, at 354, an annotated patient photograph image that represents at least a portion of the treatment plan with reference to the patient photograph provided by the patient representative, such that the explanatory resources include the annotated patient photograph. In such examples, the annotated patient photograph image may include at least a portion of the patient photograph as well as annotations indicating one or more aspects of the treatment plan. As a more specific example, the annotated patient photograph image may correspond to a patient photograph that has been annotated, marked, and/or otherwise altered to emphasize and/or clarify aspects of the patient’s mouth and/or oral condition that the treatment plan is intended to address. In such examples, the annotated patient photograph image may include and/or be any appropriate combination of photograph images and illustrated images.

As another example, and as shown in FIG. 15, the producing the one or more explanatory resources at 352 may include producing, at 356, a target patient image that represents an anticipated appearance of the patient’s mouth as effected, affected, and/or otherwise expected to be produced by the treatment plan, such that the explanatory resources include the target patient image. In such examples, the target patient image may include and/or be a version of the patient photograph that has been modified, such as with an image editing computer program, to simulate, depict, visualize, and/or otherwise represent a potential appearance of the patient’s mouth, jaws, teeth, etc. as resulting from execution of the treatment plan. In this manner, providing the patient representative with the target patient image may enhance the patient’s willingness and/or enthusiasm to pursue the treatment plan upon witnessing the potential benefits of the treatment plan as applied to their own appearance. In such examples, the target patient image may include and/or be any appropriate combination and/or sequences of photograph images and/or illustrated images.

In some examples, and as shown in FIG. 15, the producing the one or more explanatory resources at 352 includes identifying, at 358, one or more treatment plan media files from a plurality of treatment plan media files, such that the explanatory resources include the treatment plan media files. Each treatment plan media file may include and/or be any appropriate media files and/or resources for conveying at least a portion and/or an aspect of the treatment plan to the patient representative, such as treatment plan media file 190 schematically illustrated in FIG. 1. As examples, each diagnostic media reference may include and/or be a video file showing and/or describing an aspect of the treatment plan, an image and/or photograph showing and/or describing an aspect of the treatment plan, and/or an audio file representing and/or describing an aspect of the treatment plan. As a more specific example, FIG. 14 illustrates an example of user interface 76, such as may be produced by centralized service 70 and/or displayed on the patient electronic device, that displays an explanatory resource 184 in the form of a treatment plan media file 190 describing aspects of an oral condition. In this manner, providing the patient representative with one or more treatment plan media files may serve to supplement the patient representative’s comprehension of the treatment plan and/or willingness to enact the treatment plan upon the patient.

In some examples, and as shown in FIG. 15, the identifying the treatment plan media file(s) at 358 includes accessing, at 360, a media resource library that includes the plurality of treatment plan media files. In some examples, and as schematically illustrated in FIG. 1, media resource library 180 may be at least partially stored by centralized service 70 and/or by service provider electronic device 80. Accordingly, the accessing the media resource library at 360 may be performed with the service provider electronic device, such as to access the media resource library directly and/or via the centralized service.

In some examples, and as shown in FIG. 15, method 300 further includes, subsequent to the generating the treatment plan at 340, conveying, at 370, at least a portion of the treatment plan to the patient representative. The conveying the treatment plan to the patient representative at 370 may include conveying a portion of the treatment plan that is most relevant to the patient representative. For example, the conveying the treatment plan to the patient representative at 370 may include conveying the treatment plan framework but not all of the treatment plan details. Additionally or alternatively, the conveying the treatment plan at 370 may include conveying one or more explanatory resources that were produced by the producing the explanatory resource(s) at 352. The conveying the treatment plan at 370 may include conveying the treatment plan directly to the patient representative, and/or may include conveying the treatment plan to the patient representative via the centralized service (e.g., such that the patient representative receives the treatment plan via the patient electronic device). In such examples, the patient representative may have the opportunity to review the treatment plan as proposed by the service provider to evaluate whether to pursue the treatment plan. In some examples, the conveying the treatment plan to the patient representative at 370 includes directing the patient representative to access the media resource library, such as to direct and/or enable the patient representative to access one or more explanatory resources with the patient electronic device. In this manner, the conveying the treatment plan to the patient representative at 370 may include conveying aspects of the treatment plan (such as the explanatory resources) to the patient representative without delivering such aspects directly to the patient representative.

In some examples, and as shown in FIG. 15, method 300 further includes, subsequent to the generating the treatment plan at 340, enacting, at 390, the treatment plan to treat the oral condition of the patient. In some such examples, method 300 additionally includes, subsequent to the conveying the treatment plan to the patient representative at 370, obtaining approval, at 380, of the treatment plan from the patient representative. Specifically, in such examples, the enacting the treatment plan at 390 is performed subsequent to, and responsive to, the obtaining the approval of the treatment plan at 380. The obtaining approval of the treatment plan at 380 may be performed in any appropriate manner, such as by obtaining a signature and/or other confirmation of consent by the patient representative for the service provider to enact the treatment plan. In some examples, the obtaining approval of the treatment plan at 380 additionally or alternatively includes receiving a payment from the patient representative, and/or otherwise on behalf of the patient (e.g., via an insurance provider), prior to the enacting the treatment plan at 390.

The enacting the treatment plan at 390 may be performed by any appropriate entity and may include any appropriate procedures. In some examples, at least a portion of the enacting the treatment plan at 390 is performed by the service provider. In some examples, the enacting the treatment plan at 390 includes performing a measurement of at least a portion of the patient’s mouth, such as by photographing the patient’s mouth, forming an X-ray image of the patient’s mouth, forming a physical mold of the patient’s mouth, and/or forming a computer-generated model of the patient’s mouth. In this manner, the performing the measurement of the patient’s mouth may serve to confirm, correct, and/or supplement the oral condition data set by performing measurements that utilize equipment and/or operational expertise that is not available to the patient representative alone.

The enacting the treatment plan at 390 may include performing any appropriate actions relative to the patients mouth. As examples, the treatment plan may correspond to an orthodontic treatment, and the enacting the treatment plan at 390 may include installing an orthodontic device within the patient’s mouth, adjusting an orthodontic device positioned within the patient’s mouth, and/or removing an orthodontic device that is positioned within the patient’s mouth. As more specific examples, such methods may pertain to an orthodontic device in the form of an orthodontic bracket, an orthodontic wire, an elastic band, a headgear, a palatal expander, a spacer, and/or a retainer. As additional examples, the treatment plan may correspond to a dental treatment, and the enacting the treatment plan may include installing a dental device within the patient’s mouth, adjusting a dental device within the patient’s mouth, and/or removing a dental device that is positioned within the patient’s mouth. As more specific examples, such methods may pertain to a dental device in the form of a dental implant, a dental crown, a dental veneer, a dental sealant, a retainer, and/or a splint.

The foregoing discussion generally pertains to examples of utilizing diagnostic communication system 1000 and/or centralized service 70 to facilitate coordination of information transfer between a single patient electronic device 60 and a single service provider electronic device 80. However, this is not required, and it is additionally within the scope of the present disclosure that the systems and methods disclosed herein may be applied to any appropriate combination of patient electronic devices and service provider electronic devices. For example, diagnostic communication system 1000 and/or centralized service 70 may be configured to facilitate coordination of information transfer between a single service provider representative and each of a plurality of patient representatives. As a more specific example, and as schematically illustrated in FIG. 1, centralized service 70 may be configured to interact with each of a plurality of patient electronic devices 60 associated with a corresponding plurality of patients. Additionally or alternatively, and as further schematically illustrated in FIG. 1, centralized service 70 may be configured to interact with each of a plurality of service provider electronic devices 80 associated with a corresponding plurality of service providers. Accordingly, centralized service 70 may be configured to facilitate information transfer between a single patient electronic device 60 and a single service provider electronic device 80, between a single patient electronic device 60 and each of a plurality of service provider electronic devices 80, between each of a plurality of patient electronic devices 60 and a single service provider electronic device 80, and/or between each of a plurality of patient electronic devices 60 and each of a plurality of service provider electronic devices 80. For example, a given (e.g., a particular) service provider representative may provide data collection kit 100 to each of a plurality of patient representatives and subsequently collect a corresponding plurality of oral condition data sets 160, thus enabling the service provider to process a greater number of prospective and/or incoming patients than would be possible if performing initial consultations in person. As another example, a given (e.g., a particular) patient representative may utilize diagnostic communication system 1000 and/or centralized service 70 to deliver oral condition data set 160, or each of a plurality of unique oral condition data sets 160, to a plurality of distinct service provider representatives. In this manner, the patient representative may utilize diagnostic communication system 1000 and/or centralized service 70 to seek diagnoses for a plurality of distinct oral conditions of the patient corresponding to distinct service providers, and/or to seek a diagnosis of a given (e.g., a particular) oral condition of the patient from each of a plurality of distinct service providers.

The flowcharts and block diagrams described herein illustrate the architecture, functionality, and operation of possible implementations of systems, methods, and computer program products according to various illustrative embodiments. In this regard, each block in the flowcharts or block diagrams may represent a module, segment, or portion of code, which comprises one or more executable instructions for implementing the specified logical function or functions. It should also be noted that, in some alternative implementations, the functions noted in a block may occur out of the order noted in the drawings. For example, the functions of two blocks shown in succession may be executed substantially concurrently, or the functions of the blocks sometimes may be executed in the reverse order, depending upon the functionality involved.

In the present disclosure, several of the examples have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order and/or concurrently. It also is within the scope of the present disclosure that the blocks, or steps, may be implemented as logic, which also may be described as implementing the blocks, or steps, as logics. In some applications, the blocks, or steps, may represent expressions and/or actions to be performed by functionally equivalent circuits or other logic devices. The illustrated blocks may, but are not required to, represent executable instructions that cause a computer, processor, and/or other logic device to respond, to perform an action, to change states, to generate an output or display, and/or to make decisions.

As used herein, the term “and/or” placed between a first entity and a second entity means one of (1) the first entity, (2) the second entity, and (3) the first entity and the second entity. Multiple entries listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising,” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities, should be understood to mean at least one entity selected from any one or more of the entity in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “At least one of A or B,” or, equivalently, “at least one of A and/or B”) may refer, in one embodiment to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” may mean A alone, B alone, C alone, A and B together, A and C together, B and C together, A, B, and C together, and optionally any of the above in combination with at least one other entity.

As used herein, the phrase “at least substantially,” when modifying a degree or relationship, includes not only the recited “substantial” degree or relationship, but also the full extent of the recited degree or relationship. A substantial amount of a recited degree or relationship may include at least 75% of the recited degree or relationship. For example, a first direction that is at least substantially parallel to a second direction includes a first direction that is within an angular deviation of 22.5° relative to the second direction and also includes a first direction that is identical to the second direction.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It also is within the scope of the present disclosure that elements, components, and/or other recited subject matter that is recited as being adapted to perform a particular function may additionally or alternatively be described as being configured to perform that function, and vice versa.

As used herein, the terms “selective” and “selectively,” when modifying an action, movement, configuration, or other activity of one or more components or characteristics of an apparatus, mean that the specific action, movement, configuration, or other activity is a direct or indirect result of one or more dynamic processes, as described herein. The terms “selective” and “selectively” thus may characterize an activity that is a direct or indirect result of user manipulation of an aspect of, or one or more components of, the apparatus, or may characterize a process that occurs automatically, such as via the mechanisms disclosed herein.

As used herein, the phrase “for example,” the phrase “as an example,” and/or simply the term “example,” when used with reference to one or more components, features, details, structures, embodiments, and/or methods according to the present disclosure, are intended to convey that the described component, feature, detail, structure, embodiment, and/or method is an example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

Examples of systems, data collection kits, and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

A1. A data collection kit for generating an oral condition data set that characterizes an oral condition of a patient, the data collection kit comprising one or more of:
(i) at least one diagnostic instrument configured to facilitate characterizing at least a portion of the oral condition;
(ii) instructions describing a method of generating at least a portion of the oral condition data set; and
(iii) packaging that contains the at least one diagnostic instrument, and optionally that further contains at least a portion of the instructions.

A2. The data collection kit of paragraph A1, wherein the at least one diagnostic instrument includes one or more of:
(i) a cheek retractor configured to bias at least one of the patient’s cheeks or lips apart along an occlusal plane of the patient’s mouth, in a superior direction, or in an inferior direction;
(ii) an overjet gauge configured to provide a measurement of a maxillary overjet of the patient’s teeth, optionally the patient’s incisor teeth; and
(iii) a mouth opening gauge configured to provide a measurement of a maximum mouth opening distance of the patient’s mouth.

A3. The data collection kit of any of paragraphs A1-A2, wherein the oral condition data set includes information that pertains to one or more of the patient’s mouth, the patient’s gums, the patient’s tongue, the patient’s palate, the patient’s airway, the patient’s jaw, the patient’s maxilla, the patient’s mandible, and the patient’s teeth.

A4. The data collection kit of any of paragraphs A1-A3, wherein the oral condition data set includes one or more of orthodontic data, dental data, intraoral data, endodontic data, periodontic data, otolaryngological data, and otorhinolaryngological data.

A5. The data collection kit of any of paragraphs A1-A4, wherein the oral condition data set includes one or more of:
(i) at least one patient photograph depicting at least a portion of the patient’s mouth;
(ii) measurement data;
(iii) numerical data; and
(iv) text data.

A6. The data collection kit of any of paragraphs A2-A5, wherein the cheek retractor includes, and optionally is, a one-piece cheek retractor configured to engage both of the patient’s cheeks simultaneously.

A7. The data collection kit of any of paragraphs A2-A6, wherein the cheek retractor includes, and optionally is, one or more cheek retractor portions, each cheek retractor portion configured to engage a respective cheek of the patient.

A8. The data collection kit of any of paragraphs A2-A7, wherein the overjet gauge is configured to be placed against one or both of the patient’s maxilla, optionally an upper reference tooth of the patient’s maxilla, and the patient’s mandible, optionally a lower reference tooth of the patient’s mandible, to measure the maxillary overjet between the patient’s maxilla and the patient’s mandible.

A9. The data collection kit of any of paragraphs A2-A8, wherein the overjet gauge includes a plurality of overjet zones, optionally color-coded overjet zones, that are configured to correspond to respective discrete ranges of the overjet of the patient’s teeth.

A10. The data collection kit of any of paragraphs A2-A9, wherein the mouth opening gauge includes:
a notch configured to engage one of a/the lower reference tooth and a/the upper reference tooth; and
a scale configured to quantify a distance between the lower reference tooth and the upper reference tooth when the patient’s mouth is fully open and when the notch engages the one of the lower reference tooth and the upper reference tooth.

A11. The data collection kit of any of paragraphs A2-A10, wherein the mouth opening gauge further is configured to provide a measurement of a severity of ankyloglossia in the patient’s mouth.

A12. The data collection kit of any of paragraphs A2-A11, wherein the at least one diagnostic instrument includes an integrated gauge tool that includes each of the overjet gauge and the mouth opening gauge.

A13. The data collection kit of paragraph A12, wherein the integrated gauge tool is formed on a card, optionally a plastic card.

A14. The data collection kit of any of paragraphs A12-A13, wherein the integrated gauge tool includes the overjet gauge and the mouth opening gauge integrated with one another, and wherein the integrated gauge tool is configured to facilitate selective separation of the overjet gauge and the mouth opening gauge from one another by an end user.

A15. The data collection kit of any of paragraphs A1-A14, further comprising a tracking code for at least partially identifying the data collection kit.

A16. The data collection kit of paragraph A15, wherein the oral condition data set includes tracking code information that includes, and optionally is, one or both of the tracking code and a representation of the tracking code.

A17. The data collection kit of paragraph A16, wherein the tracking code information includes, and optionally is, one or more of a textual description of the tracking code, an image of the tracking code, and a photograph of the tracking code, optionally a photograph of the tracking code that is taken during operative use of the at least one diagnostic instrument.

A18. The data collection kit of any of paragraphs A15-A17, wherein the tracking code includes one or more of an alphanumeric sequence, a word, a sequence of words, a number, a barcode, a URL, a quick response (QR) code, one or more colors, one or more patterns, and one or more shapes.

A19. The data collection kit of any of paragraphs A15-A18, wherein the tracking code is configured to correspond to one or more of:
(i) an identity of a service provider to whom the oral condition data set is intended to be delivered;
(ii) the identity of the service provider that provides the data collection kit to the patient;
(iii) the identity of the patient;
(iv) a unique identifier of the data collection kit;
(v) a date, or date range, on which the data collection kit is provided to the patient;
(vi) a geographical region in which the data collection kit is provided to the patient; and
(vii) an identity of a marketing campaign within which the data collection kit is provided to the patient.

A20. The data collection kit of any of paragraphs A15-A19, wherein the tracking code is one or more of:
(i) printed on one or more of the at least one diagnostic instrument, the instructions, and the packaging; and
(ii) operatively affixed to one or more of the at least one diagnostic instrument, the instructions, and the packaging.

A21. The data collection kit of any of paragraphs A15-A20, wherein the tracking code is configured to be physically transferred from one component of the data collection kit to one or both of:
(i) another component of the data collection kit; and
(ii) the oral condition data set.

A22. The data collection kit of any of paragraphs A15-A21, wherein the tracking code is printed on one or more of a sticker, a decal, a stamp, and a transfer that is configured to be selectively affixed to the at least one diagnostic instrument by an end user of the data collection kit.

A23. The data collection kit of any of paragraphs A1-A22, wherein the instructions include instructions for operative use of the at least one diagnostic instrument to at least partially characterize the oral condition.

A24. The data collection kit of any of paragraphs A1-A23, wherein the packaging includes the instructions.

A25. The data collection kit of any of paragraphs A1-A24, wherein the instructions include printed instructions that are printed on one or more of the packaging, the at least one diagnostic instrument, and an instructions printout that is included within the packaging.

A26. The data collection kit of any of paragraphs A1-A25, wherein the instructions include instructions that are configured to be accessed via an Internet connection.

A27. The data collection kit of any of paragraphs A1-A26, wherein the instructions include instructions for delivering the oral condition data set to a/the service provider and/or a/the centralized service, optionally via a patient electronic device that is associated with the patient.

A28. The data collection kit of any of paragraphs A1-A27, wherein the packaging includes one or more of a container, a resealable container, a box, and a bag.

A29. The data collection kit of any of paragraphs A1-A28, wherein the packaging is configured to be shipped to the patient and/or a patient representative via a postal service.

A30. The data collection kit of any of paragraphs A1-A29, wherein one or more of the at least one diagnostic instrument are at least partially integrated with the packaging and configured to be selectively removed from the packaging prior to utilizing the at least one diagnostic instrument.

A31. The data collection kit of any of paragraphs A1-A30, wherein at least a portion of the data collection kit, and optionally all of the data collection kit, is configured to be printed by the patient and/or a/the patient representative.

A32. The data collection kit of paragraph A31, wherein the portion of the data collection kit is configured to be downloaded and printed by the patient and/or the patient representative.

B1. A method of conveying an oral condition data set regarding an oral condition of a patient to a service provider, the method comprising:
generating the oral condition data set; and
delivering the oral condition data set to the service provider or a centralized service.

B2. The method of paragraph B 1, wherein the method is performed, at least in part, by a patient representative that includes one or both of the patient and a patient proxy that represents the patient.

B3. The method of paragraph B2, wherein the patient proxy is one or more of a relative of the patient, a parent of the patient, and a caretaker of the patient.

B4. The method of any of paragraphs B 1-B3, wherein the oral condition data set is the oral condition data set obtained by utilizing the data collection kit of any of paragraphs A1-A32.

B5. The method of any of paragraphs B 1-B4, wherein the generating the oral condition data set is performed, at least in part, by utilizing the data collection kit of any of paragraphs A1-A32.

B6. The method of paragraph B5, wherein the method further includes, prior to the generating the oral condition data set, receiving the data collection kit.

B7. The method of paragraph B6, wherein the receiving the data collection kit includes receiving the data collection kit from the service provider or the centralized service.

B8. The method of any of paragraphs B5-B7, wherein at least a portion of one or both of the generating the oral condition data set and the delivering the oral condition data set is performed in accordance with the instructions.

B9. The method of any of paragraphs B1-B8, wherein the service provider is one or more of a dentist, an orthodontist, an oral surgeon, an endodontic specialist, a periodontist, an otolaryngologist, and an otorhinolaryngologist.

B10. The method of any of paragraphs B 1-B9, wherein the patient is a potential patient of the service provider.

B11. The method of any of paragraphs B1-B 10, wherein the patient is an active patient of the service provider.

B12. The method of any of paragraphs B1-B11, wherein the patient is a child younger than 7 years of age, optionally younger than 3 years of age.

B13. The method of any of paragraphs B 1-B 12, wherein the generating the oral condition data set includes measuring one or more features of the patient’s mouth.

B14. The method of paragraph B13, wherein the measuring the one or more features of the patient’s mouth includes utilizing a/the at least one diagnostic instrument of a/the data collection kit.

B15. The method of paragraph B14, wherein the measuring the one or more features of the patient’s mouth includes utilizing the data collection kit such that at least a portion of the at least one diagnostic instrument engages at least a portion of the patient’s mouth.

B16. The method of any of paragraphs B13-B15, wherein the measuring the one or more features of the patient’s mouth includes producing a measurement of a maxillary overjet of the patient’s teeth; and wherein the oral condition data set includes the measurement of the maxillary overjet of the patient’s teeth.

B17. The method of paragraph B16, when dependent from paragraph B 14, wherein the at least one diagnostic instrument includes a/the overjet gauge; and wherein the producing the measurement of the maxillary overjet is performed, at least in part, with the overjet gauge.

B18. The method of paragraph B17, wherein the overjet gauge includes a/the plurality of overjet zones; and wherein the producing the measurement of the maxillary overjet includes resting an edge of the overjet gauge against a tooth of the patient’s mandible and identifying which overjet zone of the plurality of overjet zones is proximal a tooth of the patient’s maxilla.

B19. The method of any of paragraphs B13-B18, wherein the measuring the one or more features of the patient’s mouth includes producing a measurement of a maximum mouth opening distance of the patient’s mouth, as measured between a lower reference tooth of the patient’s mandible and an upper reference tooth of the patient’s maxilla; and wherein the oral condition data set includes the measurement of the maximum mouth opening distance.

B20. The method of paragraph B19, wherein the lower reference tooth is a mandibular central incisor, and wherein the upper reference tooth is a maxillary central incisor.

B21. The method of any of paragraphs B19-B20, when dependent from paragraph B14, wherein the at least one diagnostic instrument includes a/the mouth opening gauge; and wherein the producing the measurement of the maximum mouth opening distance is performed, at least in part, with the mouth opening gauge.

B22. The method of paragraph B21, wherein the producing the measurement of the maximum mouth opening distance includes:
engaging one of the lower reference tooth and the upper reference tooth with a/the notch of the mouth opening gauge;
engaging the other of the lower reference tooth and the upper reference tooth with a/the scale of the mouth opening gauge; and
determining a position of the other of the lower reference tooth and the upper reference tooth relative to the scale when the patient’s mouth is fully open.

B23. The method of any of paragraphs B13-B22, wherein the measuring the one or more features of the patient’s mouth includes producing a measurement of a constrained maximum mouth opening distance of the patient’s mouth, as measured between a/the lower reference tooth of the patient’s mandible and a/the upper reference tooth of the patient’s maxilla; and wherein the oral condition data set includes the measurement of the constrained maximum mouth opening distance.

B24. The method of paragraph B23, wherein the producing the measurement of the constrained maximum mouth opening distance is performed while the patient maintains the patient’s tongue in contact with a lingual reference point on the roof of the patient’s mouth.

B25. The method of paragraph B24, wherein the lingual reference point is a lingual side of the patient’s mandibular central incisors.

B26. The method of any of paragraphs B23-B25, when dependent from paragraph B 14, wherein the at least one diagnostic instrument includes a/the mouth opening gauge; and wherein the producing the measurement of the constrained maximum mouth opening distance is performed, at least in part, with the mouth opening gauge.

B27. The method of paragraph B26, wherein the producing the measurement of the constrained maximum mouth opening distance includes:
engaging one of the lower reference tooth and the upper reference tooth with a/the notch of the mouth opening gauge;
engaging the other of the lower reference tooth and the upper reference tooth with a/the scale of the mouth opening gauge; and
determining a position of the other of the lower reference tooth and the upper reference tooth relative to the scale when the patient’s mouth is fully open with the patient’s tongue maintained in contact with a/the lingual reference point.

B28. The method of any of paragraphs B1-B27, wherein the generating the oral condition data set includes producing one or more patient photographs that represent at least a portion of the patient’s mouth; and wherein the oral condition data set includes the one or more patient photographs.

B29. The method of paragraph B28, wherein the producing the one or more patient photographs includes retrieving one or more preexisting photographs that represent at least a portion of the patient’s mouth.

B30. The method of any of paragraphs B28-B29, wherein the producing the one or more patient photographs includes photographing with a camera associated with the patient.

B31. The method of paragraph B30, wherein the photographing includes photographing at least a portion of the patient’s mouth while biasing the patient’s cheeks or lips apart along the occlusal plane of the patient’s mouth, or in superior and inferior directions, optionally with a/the cheek retractor.

B32. The method of any of paragraphs B30-B31, wherein the photographing includes photographing the patient’s airway.

B33. The method of any of paragraphs B30-B32, wherein the photographing includes photographing an underside of the patient’s tongue.

B34. The method of any of paragraphs B30-B33, wherein the photographing includes photographing the patient’s teeth with the patient’s jaw closed.

B35. The method of any of paragraphs B30-B34, wherein the photographing includes photographing such that at least a portion of a/the tracking code is visible in the patient photograph.

B36. The method of any of paragraphs B1-B35, wherein the delivering the oral condition data set includes delivering at least a portion of the oral condition data set directly to the service provider.

B37. The method of paragraph B36, wherein the delivering at least a portion of the oral condition data set directly to the service provider includes physically bringing at least a portion of the oral condition data set to the service provider.

B38. The method of any of paragraphs B36-B37, wherein the delivering directly to the service provider includes sending at least a portion of the oral condition data set to the service provider via one or more of:
(i) electronic mail;
(ii) a text message;
(iii) a telephone call; and
(iv) a postal service.

B39. The method of any of paragraphs B1-B38, wherein the delivering the oral condition data set includes delivering at least a portion of the oral condition data set via the centralized service.

B40. The method of any of paragraphs B1-B39, wherein the delivering the oral condition data set includes:
inputting the oral condition data set with a/the patient electronic device associated with the patient; and
transmitting, with the patient electronic device, the oral condition data set to one or both of the service provider and the centralized service.

B41. The method of paragraph B40, wherein the patient electronic device includes, and optionally is, one or more of an Internet-connected device, a desktop computer, a laptop computer, a tablet computer, a mobile electronic device, a handheld electronic device, and a cellular/smart phone.

B42. The method of any of paragraphs B40-B41, when dependent from paragraph B30, wherein the patient electronic device includes the camera.

B43. The method of any of paragraphs B40-B42, wherein a/the data collection kit includes a/the tracking code; wherein the oral condition data set includes a/the tracking code information; and wherein the inputting the oral condition data set with the patient electronic device includes inputting the tracking code information into the patient electronic device.

B44. The method of paragraph B43, wherein the inputting the tracking code information with the patient electronic device includes one or both of manually inputting the tracking code information and uploading a/the patient photograph that includes an image of at least a portion of the tracking code.

B45. The method of any of paragraphs B43-B44, when dependent from paragraph B39, wherein the inputting the oral condition data set includes communicating with the centralized service using the patient electronic device.

B46. The method of paragraph B45, wherein one or both of the communicating with the centralized service or a/the delivering via the centralized service is performed, at least in part, via an Internet connection.

B47. The method of any of paragraphs B45-B46, wherein the communicating with the centralized service includes accessing, with the patient electronic device, one or both of a Web page associated with the centralized service and a mobile application associated with the centralized service.

B48. The method of any of paragraphs B40-B47, wherein the inputting the oral condition data set includes uploading a/the one or more patient photographs to the centralized service with the patient electronic device.

B49. The method of any of paragraphs B40-B48, wherein the inputting the oral condition data set includes entering, with the patient electronic device, at least a portion of the oral condition data set into an input module generated by the centralized service.

B50. The method of paragraph B49, wherein the input module is configured to permit a user to select at least one option of a plurality of options, and wherein the inputting the oral condition data set includes entering into the input module by selecting the at least one option.

C1. A method of developing a treatment plan to be performed by a service provider to treat an oral condition of a patient, the method comprising:
receiving an oral condition data set that characterizes the oral condition;
analyzing the oral condition data set; and
generating the treatment plan;
wherein the generating the treatment plan is based, at least in part, on the analyzing the oral condition data set.

C2. The method of paragraph C1, wherein the oral condition data set is at least partially generated by the data collection kit of any of paragraphs A1-A32.

C3. The method of any of paragraphs C1-C2, wherein the method is performed responsive to the method of any of paragraphs B1-B50.

C4. The method of any of paragraphs C1-C3, wherein the method is performed, at least in part, by a service provider representative that includes one or both of the service provider and a service provider proxy that represents the service provider.

C5. The method of paragraph C4, wherein the service provider proxy is one or more of a colleague of the service provider, an assistant to the service provider, and a supervisor of the service provider.

C6. The method of any of paragraphs C1-C5, wherein the method is performed, at least in part, by a/the centralized service.

C7. The method of any of paragraphs C1-C6, wherein the receiving the oral condition data set includes receiving with a service provider electronic device associated with the service provider.

C8. The method of paragraph C7, wherein the service provider electronic device includes, and optionally is, one or more of an Internet-connected device, a desktop computer, a laptop computer, a tablet computer, a mobile electronic device, a handheld electronic device, and a cellular/smart phone.

C9. The method of any of paragraphs C7-C8, wherein the receiving the oral condition data set includes communicating with a/the centralized service using the service provider electronic device.

C10. The method of any of paragraphs C1-C9, wherein the receiving the oral condition data set is performed, at least in part, by a/the centralized service.

C11. The method of any of paragraphs C1-C10, further comprising, prior to the receiving the oral condition data set, facilitating generation of the oral condition data set.

C12. The method of paragraph C11, wherein the facilitating generation of the oral condition data set includes one or both of:
(i) providing a/the data collection kit to a/the patient representative; and
(ii) providing data collection instructions to the patient representative.

C13. The method of paragraph C12, wherein the providing the data collection instructions includes providing instructions for performing the methods of any of paragraphs B1-B50.

C14. The method of any of paragraphs C12-C13, wherein the providing the data collection kit to the patient representative is based, at least in part, on a/the tracking code information corresponding to a different data collection kit.

C15. The method of any of paragraphs C4-C14, wherein the analyzing the oral condition data set is performed, at least in part, by the service provider representative.

C16. The method of any of paragraphs C1-C15, wherein the analyzing the oral condition data set is performed, at least in part, by an automated process, optionally by an automated process of a/the centralized service.

C17. The method of any of paragraphs C1-C16, wherein the oral condition data set includes a/the patient photograph of at least a portion of the patient’s mouth, and wherein the analyzing the oral condition data set includes one or more of:
(i) initializing the patient photograph;
(ii) identifying one or more visual reference points of the patient photograph;
(iii) measuring one or more diagnostic indicators from the patient photograph, optionally as measured with reference to the one or more visual reference points; and
(iv) comparing the patient photograph to one or more diagnostic reference images.

C18. The method of paragraph C17, wherein the initializing the patient photograph includes one or more of:
(i) cropping the patient photograph;
(ii) rescaling one or more dimensions of the patient photograph to a standardized dimension; and
(iii) rotating the patient photograph to a standardized orientation.

C19. The method of any of paragraphs C17-C18, wherein the comparing the patient photograph to the one or more diagnostic reference images includes:
(i) accessing a media resource library that includes a plurality of diagnostic reference images; and
(ii) identifying the one or more diagnostic reference images from among the plurality of diagnostic reference images based, at least in part, upon a similarity to the patient photograph.

C20. The method of paragraph C19, wherein each of the one or more diagnostic reference images includes, and optionally is, at least one of a photograph, an illustration, an animation, and a video.

C21. The method of any of paragraphs C1-C20, wherein the analyzing the oral condition data set includes evaluating a Mallampati score corresponding to the patient.

C22. The method of any of paragraphs C1-C21, wherein the generating the treatment plan includes generating a procedure for treating the oral condition that includes one or more of cross bite, dental crowding, narrow arches, underbite, overbite, dead teeth, missing teeth, early tooth loss, asymmetrical dental growth, dental midline discrepancies, ankyloglossia, and dental malocclusion.

C23. The method of any of paragraphs C4-C22, wherein the generating the treatment plan is performed, at least in part, by the service provider representative.

C24. The method of paragraph C23, wherein the analyzing the oral condition data set is performed, at least in part, by an automated process, and wherein the generating the treatment plan is performed by the service provider representative based, at least in part, on the portion of the analyzing the oral condition data set that is performed by the automated process.

C25. The method of any of paragraphs C1-C24, wherein the generating the treatment plan is performed, at least in part, by an automated process, optionally by an automated process of a/the centralized service.

C26. The method of any of paragraphs C1-C25, wherein the generating the treatment plan includes:
(i) generating a treatment plan framework; and
(ii) generating treatment plan details;
wherein the treatment plan framework includes one or more general treatment plan steps; and wherein the treatment plan details include one or more specific substeps for accomplishing the one or more general treatment plan steps.

C27. The method of paragraph C26, wherein the generating the treatment plan framework is performed, at least in part, by a/the service provider representative; and wherein the generating the treatment plan details is performed, at least in part, by an automated process, optionally by an automated process of a/the centralized service.

C28. The method of any of paragraphs C26-C27, wherein the generating the treatment plan framework is performed, at least in part, by an automated process, optionally by an automated process of a/the centralized service; and wherein the generating the treatment plan details is performed, at least in part, by a/the service provider representative.

C29. The method of any of paragraphs C1-C28, wherein the generating the treatment plan includes identifying a model treatment plan from a plurality of preexisting treatment plans.

C30. The method of paragraph C29, wherein the generating the treatment plan further includes customizing the model treatment plan for the oral condition of the patient.

C31. The method of any of paragraphs C1-C30, wherein the generating the treatment plan includes independently formulating the treatment plan.

C32. The method of any of paragraphs C1-C31, wherein the treatment plan includes one or more proposed medical operations for treating the oral condition.

C33. The method of paragraph C32, wherein the treatment plan includes a proposed itinerary for performing the one or more proposed medical operations.

C34. The method of any of paragraphs C1-C33, wherein the generating the treatment plan includes producing one or more explanatory resources for describing at least a portion of the treatment plan to a/the patient representative.

C35. The method of paragraph C34, wherein the treatment plan includes the one or more explanatory resources.

C36. The method of any of paragraphs C34-C35, wherein the one or more explanatory resources includes one or more of:
(i) an annotated patient photograph image that includes at least a portion of a/the patient photograph and annotations indicating one or more aspects of the treatment plan;
(ii) a target patient image that represents an anticipated appearance of at least a portion of the patient’s mouth as effected and/or affected by the treatment plan; and
(iii) one or more treatment plan media files describing at least an aspect of the treatment plan.

C37. The method of paragraph C36, wherein the target patient image includes at least one of a photograph, an illustration, an animation, and a video.

C38. The method of any of paragraphs C36-C37, wherein each of the one or more treatment plan media files includes one or more of:
(i) a video file showing and/or describing at least an aspect of the treatment plan;
(ii) an image showing and/or describing at least an aspect of the treatment plan; and
(iii) an audio file representing and/or describing at least an aspect of the treatment plan.

C39. The method of any of paragraphs C36-C38, wherein the producing the one or more explanatory resources includes producing the annotated patient photograph image.

C40. The method of paragraph C39, wherein the producing the annotated patient photograph image includes modifying the patient photograph.

C41. The method of any of paragraphs C36-C40, wherein the producing the one or more explanatory resources includes producing the target patient image.

C42. The method of paragraph C41, wherein the producing the target patient image includes modifying the patient photograph.

C43. The method of any of paragraphs C36-C42, wherein the producing the one or more explanatory resources includes identifying the one or more treatment plan media files from a plurality of treatment plan media files.

C44. The method of paragraph C43, wherein the identifying the one or more treatment plan media files includes accessing a/the media resource library that includes the plurality of treatment plan media files and/or a plurality of diagnostic media resources.

C45. The method of paragraph C44, wherein the accessing the media resource library is performed with a/the service provider electronic device.

C46. The method of paragraph C45, wherein at least a portion of the media resource library is stored by the service provider electronic device.

C47. The method of any of paragraphs C44-C46, wherein at least a portion of the media resource library is stored by a/the centralized service.

C48. The method of any of paragraphs C1-C47, further comprising, subsequent to the generating the treatment plan, conveying at least a portion of the treatment plan to a/the patient representative.

C49. The method of paragraph C48, wherein the conveying the treatment plan includes conveying via a/the centralized service.

C50. The method of any of paragraphs C48-C49, wherein the conveying the treatment plan includes conveying directly to the patient representative.

C51. The method of any of paragraphs C1-C50, further comprising, subsequent to the generating the treatment plan, enacting the treatment plan to treat the oral condition of the patient.

C52. The method of paragraph C51, wherein the enacting the treatment plan is performed, at least in part, by the service provider.

C53. The method of any of paragraphs C51-C52, wherein the enacting the treatment plan includes performing a measurement of at least a portion of the patient’s mouth.

C54. The method of paragraph C53, wherein the performing the measurement of the patient’s mouth includes one or more of:
(i) photographing at least a portion of the patient’s mouth;
(ii) forming an X-ray image of at least a portion of the patient’s mouth;
(iii) forming a mold of at least a portion of the patient’s mouth; and
(iv) forming a computer-generated model of at least a portion of the patient’s mouth.

C55. The method of any of paragraphs C51-C54, wherein the enacting the treatment plan includes one or more of:
(i) installing an orthodontic device within the patient’s mouth;
(ii) adjusting an orthodontic device positioned within the patient’s mouth; and
(iii) removing an orthodontic device positioned within the patient’s mouth.

C56. The method of paragraph C55, wherein the orthodontic device includes one or more of an orthodontic bracket, an orthodontic wire, an elastic band, a headgear, a palatal expander, a spacer, and a retainer.

C57. The method of any of paragraphs C51-C56, wherein the enacting the treatment plan includes one or more of:
(i) installing a dental device within the patient’s mouth;
(ii) adjusting a dental device within the patient’s mouth; and
(iii) removing a dental device positioned within the patient’s mouth.

C58. The method of paragraph C57, wherein the dental device includes one or more of a dental implant, a dental crown, a dental veneer, a dental sealant, a retainer, and a splint.

C59. The method of any of paragraphs C51-C58, further comprising, subsequent to a/the conveying the at least a portion of the treatment plan to a/the patient representative, obtaining approval of the treatment plan from the patient representative; and wherein the enacting the treatment plan is performed subsequent to the obtaining approval of the treatment plan.

D1. A centralized service for coordinating information transfer between a patient and a service provider; wherein the centralized service is configured to one or more of:
(i) provide the data collection kit of any of paragraphs A1-A32 to a patient, a/the patient representative, and/or a service provider;
(ii) receive the oral condition data set of any of paragraphs B1-B50 from the patient and/or the patient representative;
(iii) facilitate the delivering the oral condition data set of any of paragraphs B1-B50 from the patient to the service provider;
(iv) analyze the oral condition data set of any of paragraphs B1-B50 to generate at least a portion, and optionally all of, the treatment plan of any of paragraphs C1-C59; and
(v) facilitate the conveying the treatment plan of any of paragraphs C1-C59 from the service provider to the patient.

D2. The centralized service of paragraph D1, wherein the centralized service is configured to perform at least a portion of the method of any of paragraphs C1-C59.

D3. The centralized service of any of paragraphs D1-D2, wherein the centralized service is configured to convey at least a portion of a respective oral condition data set from each of a plurality of patients to one or more corresponding service providers.

D4. The centralized service of any of paragraphs D1-D3, wherein the centralized service is configured to generate at least one of (i) a user interface to be presented to a/the patient representative, optionally via a/the patient electronic device, for enabling and/or facilitating the patient representative performing the method of any of paragraphs B1-B50; and (ii) a user interface to be presented to the service provider, optionally via a/the service provider electronic device, for enabling and/or facilitating the service provider performing the method of any of paragraphs C1-C59.

D5. The centralized service of any of paragraphs D1-D4, wherein the centralized service includes at least a portion of a/the media resource library.

D6. The centralized service of any of paragraphs D1-D5, wherein the centralized service includes a computer program that is configured to be executed on one or both of a/the patient electronic device and a/the service provider electronic device.

D7. The centralized service of any of paragraphs D1-D6, wherein the centralized service includes a computer program that is configured to be executed on an electronic device that is spatially removed from each of a/the patient electronic device and a/the service provider electronic device.

### INDUSTRIAL APPLICABILITY

The systems, diagnostic kits, and methods disclosed herein are applicable to the dental and orthodontic care industries.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements, and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A data collection kit for generating an oral condition data set that characterizes an oral condition of a patient, the data collection kit comprising one or more of:
(i) at least one diagnostic instrument configured to facilitate characterizing at least a portion of the oral condition;
(ii) instructions describing a method of generating at least a portion of the oral condition data set utilizing the at least one diagnostic instrument; and
(iii) packaging that contains the at least one diagnostic instrument;
wherein the instructions include instructions for delivering the oral condition data set to a service provider or a centralized service; and
wherein the method includes measuring one or more features of the patient’s mouth utilizing the at least one diagnostic instrument.

2. The data collection kit of claim 1, wherein the at least one diagnostic instrument includes an overjet gauge configured to provide a measurement of a maxillary overjet of the patient’s teeth; wherein the measuring the one or more features of the patient’s mouth includes producing the measurement of the maxillary overjet of the patient’s teeth; and wherein the oral condition data set includes the measurement of the maxillary overjet of the patient’s teeth.

3. The data collection kit of claim 2, wherein the at least one diagnostic instrument includes a cheek retractor configured to bias the patient’s cheeks or lips apart along an occlusal plane of the patient’s mouth or in superior and inferior directions.

4. The data collection kit of claim 1, wherein the at least one diagnostic instrument includes a tooth opening gauge configured to provide a measurement of a maximum mouth opening distance of the patient’s mouth; wherein the measuring the one or more features of the patient’s mouth includes producing the measurement of the maximum mouth opening distance of the patient’s mouth; and wherein the oral condition data set includes the measurement of the maximum mouth opening distance of the patient’s mouth.

5. The data collection kit of claim 4, wherein the at least one diagnostic instrument includes a cheek retractor configured to bias the patient’s cheeks or lips apart along an occlusal plane of the patient’s mouth or in superior and inferior directions.

6. The data collection kit of claim 4, wherein the tooth opening gauge is an integrated gauge tool that further includes an overjet gauge configured to provide a measurement of a maxillary overjet of the patient’s teeth; wherein the measuring the one or more features of the patient’s mouth includes producing the measurement of the maxillary overjet of the patient’s teeth; and wherein the oral condition data set includes the measurement of the maxillary overjet of the patient’s teeth.

7. The data collection kit of claim 1, wherein the method includes producing one or more patient photographs that represent at least a portion of the patient’s mouth; and wherein the oral condition data set includes the one or more patient photographs.

8. The data collection kit of claim 7, wherein the method includes producing one or more patient photographs that include at least a portion of a tracking code associated with one or more of the data collection kit and the patient.

9. The data collection kit of claim 8, wherein the tracking code is on a diagnostic instrument of the at least one diagnostic instrument; and wherein the producing includes producing a photograph of the tracking code on the diagnostic instrument while the diagnostic instrument is in the patient’s mouth.

10. The data collection kit of claim 1, wherein the instructions include instructions that are printed on at least one of the at least one diagnostic instrument, the packaging, and an insert that is included with the data collection kit.

11. The data collection kit of claim 1, wherein the instructions include at least one of instructions that are configured to be accessed via an internet connection and instructions that are configured to be accessed with a patient electronic device.

12. The data collection kit of claim 1, wherein the instructions include instructions for delivering the oral condition data set to a service provider or a centralized service.

13. The data collection kit of claim 1, further comprising a tracking code for at least partially identifying the data collection kit; and wherein the oral condition data set includes tracking code information that includes one or both of the tracking code and a representation of the tracking code.

14. The data collection kit of claim 13, wherein the tracking code is on at least one of the packaging and at least one of the at least one diagnostic instrument.

15. A method of developing a treatment plan to be performed by a service provider to treat an oral condition of a patient, the method comprising:
receiving an oral condition data set that characterizes the oral condition;
analyzing the oral condition data set; and
generating the treatment plan;
wherein the oral condition data set is generated, at least in part, by a patient representative utilizing the data collection kit of claim 1; wherein the generating the treatment plan is based, at least in part, on the analyzing the oral condition data set; and wherein the method is performed, at least in part, by a service provider representative that includes one or both of the service provider and a service provider proxy that represents the service provider.

16. The method of claim 15, wherein the receiving the oral condition data set includes receiving with a service provider electronic device associated with the service provider; and wherein the receiving the oral condition data set includes communicating with a centralized service using the service provider electronic device.

17. The method of claim 15, wherein the oral condition data set includes a patient photograph of at least a portion of the patient’s mouth, and wherein the analyzing the oral condition data set includes one or more of:
(i) initializing the patient photograph;
(ii) identifying one or more visual reference points of the patient photograph;
(iii) measuring one or more diagnostic indicators from the patient photograph, optionally as measured with reference to the one or more visual reference points; and
(iv) comparing the patient photograph to one or more diagnostic reference images.

18. The method of claim 15, wherein the generating the treatment plan is performed, at least in part, by the service provider representative; and wherein the generating the treatment plan is performed, at least in part, by an automated process.

19. The method of claim 15, wherein the generating the treatment plan includes producing one or more explanatory resources for describing at least a portion of the treatment plan to the patient representative; wherein the one or more explanatory resources includes an annotated patient photograph image that includes at least a portion of a patient photograph of at least a portion of the patient’s mouth and annotations indicating one or more aspects of the treatment plan; and wherein the treatment plan includes the one or more explanatory resources.

20. The method of claim 15, wherein the generating the treatment plan includes producing one or more explanatory resources for describing at least a portion of the treatment plan to the patient representative; wherein the one or more explanatory resources includes a target patient image that represents an anticipated appearance of at least a portion of the patient’s mouth as effected by the treatment plan; and wherein the treatment plan includes the one or more explanatory resources.
